(19) United States
(12) Patent Application Publication
(10) Pub. No.: US 2023/0416315 A1
(43) Pub. Date: Dec. 28, 2023

(54) PRION-FC REGION FUSION PROTEIN AND USE THEREOF

(71) Applicant: TOOLGEN INCORPORATED, Seoul (KR)

(72) Inventors: Sang Woo HAM, Seoul (KR); Jae Young LEE, Seoul (KR)

(21) Appl. No.: 18/031,202

(22) PCT Filed: Nov. 3, 2021
(86) PCT No.: PCT/KR2021/015775

§ 371 Date: Apr. 11, 2023

(30) Foreign Application Priority Data
Nov. 3, 2020 (KR) 10-2020-0145568

(51) Int. Cl. C07K 14/47; A61K 38/17; A61P 25/28; C12N 15/86

(52) U.S. Cl. CPC C07K 14/47; A61K 38/1709; A61P 25/28; C12N 15/86; C07K 2319/30; C07K 2319/02; A61K 48/00

(57) ABSTRACT

The present invention relates to prion-Fc region fusion proteins and vectors capable of expressing the prion-Fc region fusion proteins. More particularly, the prion-Fc region fusion protein is a fusion protein comprising the Fc region of a human prion protein and an immunoglobulin. The prion-Fc region fusion protein can function to act on and remove the neurotoxic protein aggregates described above. In addition, the present invention relates to a use the prion-Fc region fusion proteins, or vectors capable of expressing the same, and a method for treating diseases associated with neurotoxic protein aggregates.

### TECHNICAL FIELD

The present invention relates to in the field of technology for the treatment of neurodegenerative diseases.

### BACKGROUND ART

Neurodegenerative diseases are diseases in which the nerve cells of the central nervous system deteriorate over time, resulting in dysfunction and disability. Patients with neurodegenerative diseases exhibit a wide range of dysfunctions, including motor control, cognitive, perceptual, sensory, and other autonomic functions.

These neurodegenerative diseases are classified by the area of the brain where the neuronal abnormalities occur and by their primary symptoms, and include Alzheimer's disease, Parkinson's disease, Lewy bodies disease, Pick's disease, Traumatic encephalopathy, Amyotrophic Lateral Sclerosis; ALS), Tauopathy, and Frontotemporal dementia.

The pathogenesis of neurodegenerative diseases is not clearly discovered, and it is understood to be caused by various mechanisms such as oxidative damage and free radical injury, mitochondrial dysfunction, and energy failure, axonal transport defects in neuronal axons, neuroinflammation, neuronal apoptosis, intracellular aggregates accumulation, protein oligomer toxicity, and abnormal protein degradation system dysfunction. Among these mechanisms, intracellular accumulation, and propagation of aggregated proteins with neurotoxicity and neuronal cell death due to the toxicity of these protein aggregates are considered to be one of the important mechanisms.

The substances associated with the above neurodegenerative diseases are mainly proteins, which are not toxic in the case of monomers synthesized physiologically in the body. When the proteins misfold in vivo to form isotypes, or when they accumulate and aggregate in the body to form oligomers, protofibrils, fibrils, and/or Lewy bodies, they become neurotoxic and are known to be closely associated with the development of neurodegenerative diseases. Representative proteins that exhibit neurotoxicity when forming protein aggregates include alpha-synuclein, amyloid beta, tau, TDP-43, and prions.

Strategies to prevent the accumulation of these neurotoxic protein aggregates and to remove those that have already accumulated have been proposed as important strategies to treat neurodegenerative diseases.

### DISCLOSURE

### Technical Problem

An objective of the present disclosure is to provide prion-Fc region fusion proteins capable of inducing clearance of neurotoxic protein aggregates.

Another objective of the present disclosure is to provide a vector capable of expressing the prion-Fc region fusion proteins.

A further objective of present disclosure is to provide compositions comprising the prion-Fc region fusion protein or a vector capable of expressing the prion-Fc region fusion protein.

A further objective of present disclosure is to provide uses for compositions comprising the prion-Fc region fusion protein or a vector capable of expressing the prion-Fc region fusion protein.

A further objective of present disclosure is to provide methods of treating neurodegenerative diseases using a composition comprising the prion-Fc region fusion protein or a vector capable of expressing the prion-Fc region fusion protein.

A further objective of present disclosure is to provide methods for removing the neurotoxic protein aggregates using a composition comprising the prion-Fc region fusion protein or a vector capable of expressing the prion-Fc region fusion protein.

### Technical Solution

The present invention provides a fusion protein capable of inducing the removal of neurotoxic protein aggregates represented by the following [Formula 1]:

hPrP−L1−Fc [Formula 1]

wherein the hPrP is selected from a group of full-length human prion protein, human prion protein fragment, human prion protein variant, and fragment of human prion protein variant,
wherein the L1 is a linker or non-covalent entity,
wherein the Fc is the Fc region of human or mouse immunoglobulin G, and
wherein the neurotoxic protein aggregates are one or more selected from a group of alpha-synuclein aggregates, amyloid beta aggregates, tau aggregates, TDP-43 aggregates, and prion aggregates.

In one embodiment, the hPrP provides a fusion protein represented by a sequence selected from the following sequences:

(SEQ ID NO. 1)
MANLGCWMLVLFVATWSDLGLCKKRPKPGGWNTGGSRYPGQGSPGGNR
YPPQGGGGWGQPHGGGWGQPHGGGWGQPHGGGWGQPHGGGWGQGGGTHS
QWNKPSKPKTNMKHMAGAAAAGAVVGGLGGYMLGSAMSRPIIHFGSDYE
DRYYRENMHRYPNQVYYRPMDEYSNQNNFVHDCVNITIKQHTVTTTTKG
ENFTETDVKMMERVVEQMCITQYERESQAYYQRGSSMVLFSSPPVILLI
SFLIFLIVG;
(SEQ ID NO. 30)
PQGGGGWGQPHGGGWGQPHGGGWGQPHGGGWGQPHGGGWGQGGG
THSQWNKPSKPKTNMKH;
(SEQ ID NO. 31)
PQGGGGWGQPHGGGWGQPHGGGWGQPHGGGWGQPHGGGWGQGGG
THSQWNKPSKPKTNMKHMAGAAAAGAV
VGGLGGYMLGSAM;
(SEQ ID NO. 32)
KKRPKPGGWNTGGSRYPGQGSPGGNRYPPQGGGGWGQPHGGGWGQP
HGGGWGQPHGGGWGQPHGGGWGQGG GTHSQWNKPSKPKTNMKH;
(SEQ ID NO. 33)
KKRPKPGGWNTGGSRYPGQGSPGGNRYPPQGGGGWGQPHGGGWGQP
HGGGWGQPHGGGWGQPHGGGWGQGG
GTHSQWNKPSKPKTNMKHMAGAAAAGAVVGGLGGYMLGSAM;
(SEQ ID NO. 34)
THSQWNKPSKPKTNMKHMAGAAAAGAVVGGLGGYMLGSAM;
(SEQ ID NO. 35)
MANLGCWMLVLFVATWSDLGLCKKRPKPGGWNTGGSRYPGQGSPGGNR
YPPQGGGGWGQPHGGGWGQPHGGGWGQPHGGGWGQPHGGGWGQGGGTHS
QWNKPSKPKTNMKHMAGAAAAGAVVGGLGVYMLGSAMSRPIIHFGSDYE
DRYYRENMHRYPNQVYYRPMDEYSNQNNFVHDCVNITIKQHTVTTTTKG
ENFTETDVKMMERVVEQMCITQYERESQAYYQRGSSMVLFSSPPVILLI
SFLIFLIVG;
(SEQ ID NO. 36)
PQGGGGWGQPHGGGWGQPHGGGWGQPHGGGWGQPHGGGWGQGGG
THSQWNKPSKPKTNMKHMAGAAAAGAV
VGGLGVYMLGSAM;
(SEQ ID NO. 37)
KKRPKPGGWNTGGSRYPGQGSPGGNRYPPQGGGGWGQPHGGGWGQP
HGGGWGQPHGGGWGQPHGGGWGQGG
GTHSQWNKPSKPKTNMKHMAGAAAAGAVVGGLGVYMLGSAM;
(SEQ ID NO. 38)
THSQWNKPSKPKTNMKHMAGAAAAGAVVGGLGVYMLGSAM;
(SEQ ID NO. 39)
MANLGCWMLVLFVATWSDLGLCKKRPKPGGWNTGGSRYPGQGSPGGNR
YPPQGGGGWGQPHGGGWGQPHGGGWGQPHGGGWGQPHGGGWGQGGGTHS
QWNKPSKPKTNMKHMAGAAAAGAVVGGLGGYVLGSAMSRPIIHFGSDYE
DRYYRENMHRYPNQVYYRPMDEYSNQNNFVHDCVNITIKQHTVTTTTKG
ENFTETDVKMMERVVEQMCITQYERESQAYYQRGSSMVLFSSPPVILLI
SFLIFLIVG;
(SEQ ID NO. 40)
KKRPKPGGWNTGGSRYPGQGSPGGNRYPPQGGGGWGQPHGGGWGQP
HGGGWGQPHGGGWGQGGGTHSQWNK
PSKPKTNMKHMAGAAAAGAVVGGLGGYVLGSAM;
(SEQ ID NO.41)
PQGGGGWGQPHGGGWGQPHGGGWGQPHGGGWGQPHGGGWGQGGG
THSQWNKPSKPKTNMKHMAGAAAAGAV
VGGLGGYVLGSAM;
(SEQ ID NO. 42)
THSQWNKPSKPKTNMKHMAGAAAAGAVVGGLGGYVLGSAM;
(SEQ ID NO. 43)
MANLGCWMLVLFVATWSDLGLCKKRPKPGGWNTGGSRYPGQGSPGGNR
YPPQGGGGWGQPHGGGWGQPHGGGWGQPHGGGWGQPHGGGWGQGGGTHS
QWNKPSKPKTNMKHMAGAAAAGAVVGGLGGYMLGSAMSRPIIHFGSDYE
DRYYRENMHRYPNQVYYRPMDEYSNQNNFVHDCVNITIKQHTVTTTTKG
ENFTETDVKMMERVVEQMCITQYKRESQAYYQRGSSMVLFSSPPVILLI
SFLIFLIVG;
and
(SEQ ID NO. 44)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPGK.

In one embodiment, the first linker provides a fusion protein represented by a sequence selected from the following sequences:

ISA;
(SEQ ID NO. 18)
ISAMVRS;
(G)n;
(SEQ ID NO. 19)
(GGGGS)n;
(SEQ ID NO. 20)
(EAAAK)n;
and
(XP)n.

In one embodiment, the Fc provides a fusion protein that is the Fc region of human immunoglobulin G.

In one embodiment, the human immunoglobulin G provides a fusion protein selected from a group of IgG1, IgG2, IgG3, and IgG4.

In one embodiment, the Fc provides a fusion protein represented by the sequence:

(SEQ ID NO. 16)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPGK.

In one embodiment, the fusion protein further comprises a secretion signal peptide, and C-terminus of the secretion signal peptide provides a fusion protein linked to N-terminus of the hPrP.

In one embodiment, the fusion protein further comprises a second linker, and the C-terminus of the secretion signal peptide and the N-terminus of the hPrP are conjugated by the second linker.

In one embodiment, the second linker provides a fusion protein represented by a sequence selected from the group of the following sequences:

ISA; ISAMVRS (SEQ ID NO. 18); (G)n; (GGGGS)n (SEQ ID NO. 19); (EAAAK)n (SEQ ID NO.20); and (XP)n.

In one embodiment, the fusion protein provides fusion protein represented by a sequence selected from the group of the following sequences:

(SEQ ID NO. 21)
MANLGCWMLVLFVATWSDLGLCKKRPKPGGWNTGGSRYPGQGSPGGNR
YPPQGGGGWGQPHGGGWGQPHGG
GWGQPHGGGWGQPHGGGWGQGGGTHSQWNKPSKPKTNMKHMAGAAAAGAVVG
GLGGYMLGSAMSRPIIHFGSDYE
DRYYRENMHRYPNQVYYRPMDEYSNQNNFVHDCVNITIKQHTVTTTTKGENFTETDV
KMMERVVEQMCITQYERE
SQAYYQRGSSMVLFSSPPVILLISFLIFLIVGISADKTHTCPPCPAPELLGGPSVFLFPPK
PKDTLMISRTPEVT
CVVVDVSHEDPEVKFNWYVDGVEVHNAKTKPREEQYNSTYRVVSVLTVLHQDWLNG
KEYKCKVSNKALPAPIEKT
ISKAKGQPREPQVYTLPPSREEMTKNQVSLTCLVKGFYPSDIAVEWESNGQPENNYKT
TPPVLDSDGSFFLYSKL
TVDKSRWQQGNVFSCSVMHEALHNHYTQKSLSLSPGK;
(SEQ ID NO. 22)
PQGGGGWGQPHGGGWGQPHGGGWGQPHGGGWGQPHGGGWGQGGG
THSQWNKPSKPKTNMKHMAGAAAAGAVVGGLGVYMLGSAMISADKTHTCPPCPAPE
LLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK;
(SEQ ID NO. 23)
PQGGGGWGQPHGGGWGQPHGGGWGQPHGGGWGQPHGGGWGQGGG
THSQWNKPSKPKTNMKHISADKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSVMH
EALHNHYTQKSLSLSPGK;
(SEQ ID NO. 24)
KKRPKPGGWNTGGSRYPGQGSPGGNRYPPQGGGGWGQPHGGGWGQP
HGGGWGQPHGGGWGQPHGGGWGQGGGTHSQWNKPSKPKTNMKHISADKTHTCP
PCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK;
(SEQ ID NO. 25)
KKRPKPGGWNTGGSRYPGQGSPGGNRYPPQGGGGWGQPHGGGWGQP
HGGGWGQPHGGGWGQPHGGGWGQGGGTHSQWNKPSKPKTNMKHMAGAAAAGA
VVGGLGVYMLGSAMISADKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCSVMHEALH
NHYTQKSLSLSPGK;
and
(SEQ ID NO. 26)
THSQWNKPSKPKTNMKHMAGAAAAGAVVGGLGVYMLGSAMISADKTHTC
PPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK.

In one embodiment, the fusion protein is characterized in that it is represented by a sequence selected from the group of the following sequences:

(SEQ ID NO. 36)
MYRMQLLSCIALSLALVTNSISAMVRSMANLGCWMLVLFVATWSDLGLCKK
RPKPGGWNTGGSRYPGQGSPGGNRYPPQGGGGWGQPHGGGWGQPHGGGWGQ
PHGGGWGQPHGGGWGQGGGTHSQWNKPSKPKTNMKHMAGAAAAGAVVGGLGGY
MLGSAMSRPIIHFGSDYEDRYYRENMHRYPNQVYYRPMDEYSNQNNFVHDCVNITIK
QHTVTTTTKGENFTETDVKMMERVVEQMCITQYERESQAYYQRGSSMVLFSSPPVILL
ISFLIFLIVGISADKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQK
SLSLSPGK;
(SEQ ID NO. 37)
MYRMQLLSCIALSLALVTNSISAMVRSPQGGGGWGQPHGGGWGQPHGGG
WGQPHGGGWGQPHGGGWGQGGGTHSQWNKPSKPKTNMKHMAGAAAAGAVVGG
LGVYMLGSAMISADKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHNHYT
QKSLSLSPGK;
(SEQ ID NO. 38)
MYRMQLLSCIALSLALVTNSISAMVRSPQGGGGWGQPHGGGWGQPHGGG
WGQPHGGGWGQPHGGGWGQGGGTHSQWNKPSKPKTNMKHISADKTHTCPPCPA
PELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFF
LYSKLTVDKSRWQQGNVFSCSVMHEALHNHYTQKSLSLSPGK;
(SEQ ID NO. 39)
MYRMQLLSCIALSLALVTNSISAMVRSKKRPKPGGWNTGGSRYPGQGSPG
GNRYPPQGGGGWGQPHGGGWGQPHGGGWGQPHGGGWGQPHGGGWGQGGGT
HSQWNKPSKPKTNMKHISADKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHE
ALHNHYTQKSLSLSPGK;
(SEQ ID NO. 40)
MYRMQLLSCIALSLALVTNSISAMVRSKKRPKPGGWNTGGSRYPGQGSPG
GNRYPPQGGGGWGQPHGGGWGQPHGGGWGQPHGGGWGQPHGGGWGQGGGT
HSQWNKPSKPKTNMKHMAGAAAAGAVVGGLGVYMLGSAMISADKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK;
and
(SEQ ID NO. 41)
MYRMQLLSCIALSLALVTNSISAMVRSTHSQWNKPSKPKTNMKHMAGAAAA
GAVVGGLGVYMLGSAMISADKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHE
ALHNHYTQKSLSLSPGK.

The present invention provides a DNA encoding the fusion protein, wherein the fusion protein is represented by a sequence selected from a group of SEQ ID NO. 21 to SEQ ID NO. 26, and SEQ ID NO. 36 to SEQ ID NO. 41.

The present invention provides a vector capable of expressing a prion-Fc domain fusion protein comprising; a DNA encoding the fusion protein; and Promoter.

In one embodiment, the vector may be an Adeno-Associate Virus(AAV).

The present invention provides a pharmaceutical composition for treating neurodegenerative diseases, comprising: the fusion protein or the vector of claim 14; and a pharmaceutically acceptable carrier.

In one embodiment, the neurodegenerative disease is selected from a group of Alzheimer's disease, Parkinson's disease, Lewy bodies disease, Pick's disease, Traumatic encephalopathy, Amyotrophic Lateral Sclerosis(ALS), Tauopathy, and Frontotemporal dementia.

The present invention provides a method of treating a neurodegenerative disease, comprising: a composition comprising the fusion protein or the vector, and a pharmaceutically acceptable carrier, administered to the central nervous system of a subject.

In one embodiment, the central nervous system of the subject may be a brain tissue of the subject.

In one embodiment, the brain tissue of the subject provides a method of treating selected from Substantia Nigra, Cerebral Ventricle, and Striatum.

In one embodiment, a method of treatment is provided wherein the composition is administered to the subject's central nervous system by a route selected from the intracerebral injection, and intracerebroventricular injection (ICV).

The present invention provides a method for removing neurotoxic protein aggregates in a subject comprising: a composition comprising the fusion protein or the vector, and a pharmaceutically acceptable carrier administered to the central nervous system of a subject.

In one embodiment, the central nervous system of the subject may be the brain tissue of the subject.

In one embodiment, a method for administering the composition to the central nervous system of a subject may be selected from the intracerebral injection, and intracerebroventricular injection (ICV).

The present invention provides a use of a composition for the manufacture of a medicament for treating neurodegenerative diseases, the composition comprising:
the fusion protein or the vector; and the pharmaceutically acceptable carrier.

### Advantageous Effects

According to the present invention, the prion-Fc domain fusion protein or vectors capable of expressing them can be used to prevent the accumulation, propagation, and neuronal cell death of neurotoxic protein aggregates, thereby providing therapeutic benefits for neurodegenerative diseases.

### BRIEF DESCRIPTION OF THE DRAWINGS

FIG. 1 is a schematic representation of an example vector capable of expressing a prion-Fc fusion protein.

FIG. 2 is a Western blot analysis of an AAV vector capable of expressing a prion-Fc fusion protein prepared according to Example 1.1. Wherein Mock refers to the control, pAAV-PrP-Fc refers to the AAV vector capable of expressing the prion-Fc fusion protein, and Anti-Fc refers to an antibody capable of binding to the Fc region.

FIG. 3 is a schematic representation of the experiment of Example 2. Specifically, it simulates preparing SH-SY5Y cells according to Example 1.2, dividing them into embodiments of No treat (Comparative Example 2.1), CM treat (Comparative Example 2.2), PFF treat (Comparative Example 2.3), and Pre-incubated PFF with CM (Example 2.1), and performing cell viability assessment (Experimental example 2.2) and immunofluorescence image observation (Experimental example 2.3).

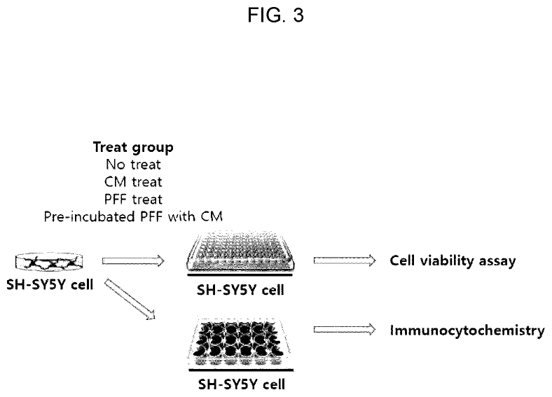

FIG. 4 shows the results of the cell viability assessment (CCK-8) according to Experimental example 2.2. Here, the vertical axis is the cell viability according to the CCK-8 assay, No treat (Ctrl) refers to Comparative Example 2.1, CM only treat refers to Comparative Example 2.2, PFF only treat refers to Comparative Example 2.3, and CM+PFF treat refers to the experimental results of Example 2.1.

FIG. 5 is an immunofluorescence image according to Experimental example 2.3. FIG. 5 shows nuclear staining results, prion-Fc region fusion protein-labeled fluorescence, and alpha-synuclein staining results. Here, No treat (Ctrl) refers to the experimental results of Comparative Example 2.1, CM only treat refers to Comparative Example 2.2, PFF only treat refers to Comparative Example 2.3, and CM+PFF treat refers to the experimental results of Example 2.1.

FIG. 6 is an immunofluorescence image according to Experimental example 2.3. FIG. 6 shows the results of alpha-synuclein staining. Here, PFF only treat refers to Comparative Example 2.3, and CM+PFF treat refers to the experimental results of Example 2.1.

FIG. 7 is the result of measuring fluorescence intensity for alpha-synuclein according to Experimental example 2.3. Here, the vertical axis represents the fluorescence intensity upon staining of alpha-synuclein, α-syn PFF/Oligomer refers to Comparative Example 2.3, α-syn PFF/Oligomer+pAAV-PrP-Fc-EGFP CM refers to the experimental results of Example 2.1.

FIG. 8 shows immunofluorescence images of the nuclei of alpha-synuclein and SIM-A9 cells, according to Experimental example 3.2. Here, PFF/Oligomer represents Comparative Examples 3.1 to 3.7 (for each incubation time), and PFF/Oligomer+pAAV—PrP-Fc-eGFP CM represents Example 3.1 to 3.7 (for each incubation time).

FIG. 9 shows immunofluorescence images of alpha-synuclein and nuclei of SIM-A9 cells, according to Experimental example 3.2. Wherein PFF represents Comparative Example 3.3, hPrP-CM+PFF represents Example 3.3, Iba1 represents immunofluorescence images of SIM-A9 cells, and pS129-α-Syn represents immunofluorescence images of alpha-synuclein.

FIG. 10 is the result of evaluating the activity against SIM-A9 cells according to Experimental example 3.3. Here, the abscissa represents the incubation time (0.5, 1, 3, 5, 8, 10, and 12 hours) and the ordinate represents the average fluorescence intensity for pS219 alpha-synuclein. PFF/Oligomer represents Comparative Example 3.3, and PFF/Oligomer+pAAV-PrP-Fc-eGFP CM represents Example 3.3.

FIG. 11 shows the average fluorescence intensity for pS219 alpha-synuclein measured inside SIM-A9 cells according to Experimental example 3.3. Here, PFF/Oligomer represents Comparative Example 3.3, and PFF/Oligomer+pAAV-PrP-Fc-eGFP CM represents Example 3.3.

FIG. 12 shows the results of a pole test on a mouse according to Experimental example 4.2. Here, the vertical axis represents the total time it took the mouse to reach the bottom of the pole. WT represents Comparative Example 4.1, LB represents Comparative Example 4.2, LB+PrP-Fc (STR) represents Example 4.1, LB+PrP-Fc (ICV) represents Example 4.2, and LB+PrP-Fc (SNpc) represents Example 4.3.

FIG. 13 shows the results of a western blot analysis of mouse brain tissue according to Experimental example 4.4.

FIG. 14 shows immunofluorescence images of mouse brain tissue according to Experimental example 4.5. Specifically, staining results for mouse dopaminergic neurons are shown. WT represents Comparative Example 4.1, LB represents Comparative Example 4.2, LB+PrP-Fc (STR) represents Example 4.1, LB+PrP-Fc (ICV) represents Example 4.2, and LB+PrP-Fc (SNpc) represents Example 4.3.

FIG. 15 shows immunofluorescence images of mouse brain tissue according to Experimental example 4.5. Specifically, staining results for alpha-synuclein are shown. WT refers to Comparative Example 4.1, LB refers to Comparative Example 4.2, and LB−PrP-Fc refers to Example 4.3.

FIG. 16 is a measurement of fluorescence intensity after immunofluorescence staining according to Experimental example 4.5. Here, the vertical axis represents the fluorescence intensity for mouse dopaminergic neurons. WT represents Comparative Example 4.1, LB represents Comparative Example 4.2, LB+PrP-Fc (STR) represents Example 4.1, LB+PrP-Fc (ICV) represents Example 4.2, and LB+PrP-Fc (SNpc) represents Example 4.3

FIG. 17 is a plot of relative TH levels from a Western blot analysis of mouse brain tissue according to Experimental example 4.4. Here, the vertical axis is the result of normalizing the TH levels of FIG. 13 as Comparative Example 4.1. WT indicates Comparative Example 4.1, LB indicates Comparative Example 4.2, and LB+PrP-Fc indicates Example 4.3.

FIG. 18 is a measurement of fluorescence intensity after immunofluorescence staining according to Experimental example 4.5. Here, the vertical axis represents the fluorescence intensity for alpha-synuclein. WT represents Comparative Example 4.1, LB represents Comparative Example 4.2, and LB+PrP-Fc represents Example 4.3.

FIG. 19 is a plot of relative pS129-α-Syn levels as a result of Western blot analysis of mouse brain tissue according to Experimental example 4.4. Here, the vertical axis is the result of normalizing the pS129-α-Syn levels of FIG. 13 to Comparative Example 4.2. WT indicates Comparative Example 4.1, LB indicates Comparative Example 4.2, and LB+PrP-Fc indicates Example 4.3.

FIG. 20 depicts relative prion protein expression levels based on Western blot analysis of mouse brain tissue according to Experimental example 4.4. Here, the vertical axis represents the expression level of hCD230 (PrP) of FIG. 13. WT indicates Comparative Example 4.1, LB indicates Comparative Example 4.2, and LB+PrP-Fc indicates Example 4.3.

FIG. 21 is a western blot analysis of the expression of human prion protein variants and fragments according to Experimental example 5.2. Here, WT indicates the expression of a prion-Fc domain fusion protein of SEQ ID NO. 21, F2 of SEQ ID NO. 22, F3 of SEQ ID NO. 23, F4 of SEQ ID NO. 24, F5 of SEQ ID NO. 25, and F6 of SEQ ID NO. 26.

FIG. 22 shows the results of evaluating in vitro efficacy against SH-SY5Y cells according to Experimental example 5.3.

FIG. 23 shows the results of a cell viability assessment (CCK-8) for primary hippocampal neuronal cells according to Experimental example 6. Here, the vertical axis represents cell viability according to the CCK-8 assay, PBS represents Comparative Example 6.1, a-syn PFF represents Comparative Example 6.3, a-syn FPFF+PrP(Full Length)-Fc represents Example 6.1, and a-syn PFF+PrP(F4)-Fc represents Example 6.2.

FIG. 24 shows the results of a cell viability assessment (CCK-8) for primary hippocampal neuronal cells according to Experimental example 6. Here, the vertical axis represents cell viability according to the CCK-8 assay, PBS represents Comparative Example 6.2, Tau PFF represents Comparative Example 6.4, Tau PFF+PrP(Full Length)-Fc represents Example 6.3, and Tau PFF+PrP(F4)-Fc represents Example 6.4.

### BEST MODE

### Definition of Terms

About

As used herein, the term “about” refers to a degree close to a certain quantity, and it refers to an amount, level, value, number, frequency, percentage, dimension, size, amount, weight, or length that varies by 30%, 25%, 20%, 15%, 10%, 9%, 8%, 7%, 6%, 5%, 4%, 3%, 2%, or 1% with respect to a reference amount, level, value, number, frequency, percentage, dimension, size, amount, weight, or length.

Subject

As used herein, the term “subject” refers to an organism that is the object of exposure to a particular substance (e.g., a peptide). The subject may refer to an independent organism, such as a human, animal, or the like, or may refer to some component of the independent organisms, such as a portion of tissue, a cell, or the like. Any of these meanings may be properly construed according to the context. In addition, the term “subject” may include any other meaning recognized by one of ordinary skill in the art.

Treatment or Cure

As used herein, the term “treatment” refers collectively to any action or measure, direct or indirect, that has the effect of eliminating, alleviating, mitigating, inhibiting, ameliorating, or preventing a condition, disease, disorder, and/or symptom in a subject. As used herein, the term “therapeutic” refers to various substances (e.g., compounds or peptides) that, when given to a subject suitably, may have a “therapeutic” effect. In addition, the terms “treatment” or “therapeutic” may have any other meaning recognized by one of ordinary skill in the art.

Standard Amino Acids

As used herein, the term “standard amino acid” refers to a group of 20 amino acids synthesized in an organism's body through the process of transcription and translation of genes. Specifically, the standard amino acids include Alanine (Ala, A), Arginine (Arg, R), Asparagine (Asn, N), Aspartic acid; Asp, D), Cysteine (Cys, C), Glutamic acid (Glu, E), Glutamine (Gin, Q), Glycine (Gly, G), Histidine; His, H), Isoleucine (Ile, I), Leucine (Leu, L), Lysine (Lys K), Methionine (Met, M), Phenylalanine (Phe, F), Proline (Pro; Pro, P), Serine (Ser, S), Threonine (Thr, T), Tryptophan (Trp, W), Tyrosine (Tyr, Y), and Valine (Val, V). Each above standard amino acid has a corresponding DNA codon and can be represented by a common amino acid mono- or tri-letter notation. The objects referred to by the term standard amino acids are to be construed as appropriate in context and include all other meanings recognizable to one of ordinary skill in the art.

Peptide Sequence Descriptions

Unless otherwise indicated, the sequences of peptides in this specification are described in the N-terminal to C-terminal direction, using amino acid monospaced or triplicated notation. For example, ISA refers to a peptide consisting of Isoleucine, Serine, and Alanine in the N-terminal to C-terminal direction. In another example, Met-Ala-Asn refers to a peptide consisting of Methionine, Alanine, and Asparagine in the N-terminal to C-terminal direction. For amino acids that cannot be represented by the above monospaced notation, other letters are used to denote them and are further described in the Supplement.

When representing a peptide in a structural formula, the N-terminus or C-terminus can be denoted using N— and —C to clarify and can be underlined to distinguish them as N-terminal and/or C-terminal. For example, when a peptide structure is written as N—B-T-A-C unless otherwise noted, the leading “N—” and trailing “—C” are symbols to clarify the N-terminal and C—
terminal orientation, meaning that the peptide is linked by a sequence represented by B, T, and A in the N-terminal to C-terminal direction.

Neurodeqenerative Disease

Overview of Neurodeqenerative Diseases

Neurodegenerative diseases are diseases in which the nerve cells of the central nervous system deteriorate over time, resulting in dysfunction and disability. Patients with neurodegenerative diseases exhibit a wide range of dysfunctions, including motor control, cognitive, perceptual, sensory, and other autonomic functions. These neurodegenerative diseases are classified by the region of the brain where the neuronal abnormalities occur and by their primary symptoms and include Alzheimer's disease, Parkinson's disease, Lewy bodies disease, Pick's disease, traumatic encephalopathy, amyotrophic lateral sclerosis (ALS), tauopathy, and amyotrophic lateral sclerosis; ALS), Tauopathy, and Frontotemporal dementia.

Pathogenesis of Neurodeqenerative Diseases

The pathogenesis of neurodegenerative diseases is not clearly discovered, and it is understood to be caused by a combination of various mechanisms such as oxidative damage and free radical injury, mitochondrial dysfunction, and energy failure, axonal transport defects in neuronal axons, neuroinflammation, neuronal apoptosis, intracellular aggregates accumulation, protein oligomer toxicity, abnormal protein degradation system dysfunction. Among these mechanisms, intracellular accumulation and propagation of aggregated proteins with neurotoxicity and neuronal cell death due to the toxicity of these protein aggregates are considered to be one of the important mechanisms.

Neurotoxic Protein Aggregates

The substances associated with the above neurodegenerative diseases are mainly proteins, which are not toxic in the case of monomers synthesized physiologically in the body. When the proteins misfold in vivo to form isotypes, or when they accumulate and aggregate in the body to form oligomers, protofibrils, fibrils, and/or Lewy bodies, they become neurotoxic and are known to be closely associated with the development of neurodegenerative diseases. Typical proteins that exhibit neurotoxicity when forming protein aggregates include alpha-synuclein, amyloid beta, tau, TDP-43, and prions, and in recent literature, several of these protein aggregates have been found simultaneously.

Strategies to prevent the accumulation of these neurotoxic protein aggregates and to remove those that have already accumulated have been proposed as important strategies to treat neurodegenerative diseases.

Limitations of Prior Arts

Limitations of Cell Therapy

One treatment for the above neurodegenerative diseases is cell therapy, which seeks to treat the central nervous system by providing healthy nerve cells from an external source. Cell therapy is a method of treating the central nervous system by differentiating stem cells of various origins (e.g., induced pluripotent stem cells, embryonic stem cells, and human unipotent stem cells) into targeted neurons (e.g., neural stem cells, neural progenitor cells, dopamine neurons, etc.) and transplanting them directly into the patient's central nervous system. This has the advantage of directly restoring the damaged central nervous system through external cell transplantation, but it is limited by 1) the difficulty of cell engraftment in the presence of protein aggregates that exhibit cytotoxicity due to the nature of degenerative brain diseases, 2) the difficulty of crossing the Blood-Brain Barrier (BBB); (BBB), which makes it difficult for external cells to penetrate the blood-brain barrier, and therefore requires direct transplantation into the brain; 3) the problem that external cells cannot proliferate and die due to undesirable nutrient supply, such as hypoxia, in the area where external cells are transplanted; and 4) the problem that the patient's immune response to external cells causes the death of external cells.

Limitations of Antibody Therapy

As another treatment for the above-mentioned neurodegenerative diseases, there is an antibody treatment therapy that attempts to prevent the accumulation of the above-mentioned protein aggregates by administering to the patient an antibody that specifically binds to the above-mentioned neurotoxic protein aggregates and removing them. Although the above antibody therapy has the advantage of acting on neurotoxic protein aggregates with high specificity, it has limitations such as 1) it is difficult to treat neurodegenerative diseases in which various types of protein aggregates are found by targeting a single protein aggregate, 2) it is difficult for the antibody to penetrate the blood-brain barrier, and 3) the antibody administered causes systemic side-effects.

Prion-Fc Region Fusion Protein

Overview of Prion-Fc Region Fusion Proteins

Disclosed herein are prion-Fc region fusion proteins. The prion-Fc region fusion protein is a fusion protein comprising a human prion protein and an Fc region of immunoglobulin. The prion-Fc region fusion protein can function to act on and remove the neurotoxic protein aggregates described above. The structure of the prion-Fc region fusion protein will be described in detail below.

Prion-Fc Region Fusion Protein Structure 1—General Representation

The prion-Fc region fusion protein comprises a human prion protein and an Fc region. The human prion protein means a whole human prion protein, a fragment of the human prion protein, a variant of the human prion protein, and/or a fragment of a variant of the human prion protein. The Fc region refers to a crystallizable fragment of a human immunoglobulin.

In one embodiment, the prion-Fc region fusion protein is a peptide sequence linked from the N terminus to the C terminus, the human prion protein and the Fc region in turn. Here, the C terminus of the human prion protein and the N terminus of the Fc region may be linked via a peptide linker.

In another embodiment, the prion-Fc region fusion protein is a peptide sequence in which, from N-terminal to C-terminal, the secretion signal peptide, the human prion protein and the Fc region are linked in turn. Here, the C-terminus of the secretion signal peptide and the N-terminus of the human prion protein, the C-terminus of the human prion protein and the N-terminus of the Fc region may each be connected via a peptide linker.

Prion-Fc Region Fusion Protein Structure 2—Structural Formula Representation

In one embodiment, the prion-Fc region fusion protein may be represented by the following [Formula 1]:

hPrP−L1−Fc [Formula 1]

Where [Formula 1] is written from the N-terminus to the C-terminus of the protein,

Wherein hPrP is a human prion protein whole, a human prion protein fragment, a human prion protein variant, and/or a fragment of a human prion protein variant,
where L is a linker, or nonexistent,

Fc is the Fc region of human immunoglobulin.

In one embodiment, the prion-Fc region fusion protein may be represented by the following [Formula 2]:

S−L1−hPrP−L2−Fc [Formula 2]

Wherein S is a Secretion Signal Peptide, or is absent, wherein L1 is the first linker, or nonexistent,

Wherein hPrP is a human prion protein whole, a human prion protein fragment, a human prion protein variant, and/or a fragment of a human prion protein variant,

The above L2 is a second linker or non-existent, Fc is the Fc region of a human immunoglobulin.

Features of Prion-Fc Region Fusion Proteins 1—Use of Prion Proteins, Fragments, or Variants Thereof

The prion-Fc region fusion protein is characterized in that it comprises a human prion protein in its entirety, a fragment thereof, and/or a variant thereof. The human prion protein is characterized in that it binds to a neurotoxic protein aggregate. The neurotoxic protein aggregates maybe, for example, oligomerized, fibrillated, or Lewy body proteins, such as α-synuclein, amyloid beta, Tau, TDP-43, and/or prions. Furthermore, the human prion protein is characterized in that it does not bind when the α-synuclein, Amyloid beta, Tau, TDP-43, and/or Prion protein is in its monomeric form—i.e., as a physiological monomer, which does not exhibit toxicity. Thus, the prion-Fc region fusion protein has the advantage of being able to selectively act on the neurotoxic protein aggregates.

Prion-Fc Region Fusion Protein Feature 2—can Act on Multiple Types of Protein Aggregates

The human prion protein is characterized in that it can bind to a plurality of the aforementioned neurotoxic protein aggregates. Thus, by targeting a plurality of neurotoxic protein aggregates, the prion-Fc region fusion protein has the advantage of targeting a plurality of protein aggregates that may appear in neurodegenerative diseases and may be effective in the treatment of various neurodegenerative diseases.

Characterization of Prion-Fc Region Fusion Proteins 3—Induction of Phagocytosis by the Fc Region

The prion-Fc region fusion protein is characterized in that it comprises the Fc region of an immunoglobulin. The Fc region functions to induce phagocytosis of macrophages, for example, macrophages and/or microglia. As described above, the human prion protein contained in the prion-Fc region fusion protein is capable of binding to neurotoxic protein aggregates, and consequently, the prion-Fc region fusion protein is capable of inducing macrophage phagocytosis of the protein aggregates, thereby effectively eliminating them.

Feature 4 of Prion-Fc Region Fusion Proteins—Increased Half-Life by the Fc Region

The Fc region can be recycled by binding to FcRn (Fc Receptor neonate) on the cell surface, which has the function of prolonging the half-life in the body. Therefore, when a drug molecule or the like is linked to the Fc region, an improved half-life in the body can be obtained than when the drug molecule is administered alone. The prion-Fc region fusion protein provided herein has the advantage of having a very long half-life in the body due to the aforementioned function of the Fc region since it is a protein in which the Fc region is fused.

Features of Prion-Fc Region Fusion Proteins 5—Prion-Fc Region Fusion Protein Expression Vectors Available

A major challenge in the treatment of neurodegenerative diseases is to get drugs across the blood-brain barrier (BBB) and into the target's brain. In general, drug molecules with large molecular weights are known to be difficult to cross the blood-brain barrier, and various strategies have been developed to do so. For drugs comprising biomaterials such as proteins, peptides, and/or nucleic acids, one strategy for crossing the blood-brain barrier is to administer an expression vector capable of crossing the blood-brain barrier. The prion-Fc region fusion proteins provided herein are characterized in that they are proteins composed of standard amino acids, and therefore nucleic acids encoding them can be constructed, and expression vectors capable of expressing the prion-Fc region fusion proteins can be constructed and used. Thus, the prion-Fc region fusion protein has the advantage that it can be produced in a vector, allowing for a variety of strategies for crossing the blood-brain barrier.

Example Prion-Fc Region Fusion Protein Sequence

In one embodiment, the prion-Fc region fusion protein may be represented by a sequence selected from the group consisting of:

(SEQ ID NO. 21)
MANLGCWMLVLFVATWSDLGLCKKRPKPGGWNTGGSRYPGQGSPGGNR
YPPQGGGGWGQPHGGGWGQPHGGGWGQPHGGGWGQPHGGGWGQGGGTHSQ
WNKPSKPKTNMKHMAGAAAAGAVVGGLGGYMLGSAMSRPIIHFGSDYEDRYYRENM
HRYPNQVYYRPMDEYSNQNNFVHDCVNITIKQHTVTTTTKGENFTETDVKMMERVVE
QMCITQYERESQAYYQRGSSMVLFSSPPVILLISFLIFLIVGISADKTHTCPPCPAPELLG
GPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK;
(SEQ ID NO. 22)
PQGGGGWGQPHGGGWGQPHGGGWGQPHGGGWGQPHGGGWGQGGG
THSQWNKPSKPKTNMKHMAGAAAAGAVVGGLGVYMLGSAMISADKTHTCPPCPAPE
LLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK;
(SEQ ID NO. 23)
QGGGGWGQPHGGGWGQPHGGGWGQPHGGGWGQPHGGGWGQGGG
THSQWNKPSKPKTNMKHISADKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSVMH
EALHNHYTQKSLSLSPGK;
(SEQ ID NO. 24)
KKRPKPGGWNTGGSRYPGQGSPGGNRYPPQGGGGWGQPHGGGWGQP
HGGGWGQPHGGGWGQPHGGGWGQGGGTHSQWNKPSKPKTNMKHISADKTHTCP
PCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK;
(SEQ ID NO. 25)
KKRPKPGGWNTGGSRYPGQGSPGGNRYPPQGGGGWGQPHGGGWGQP
HGGGWGQPHGGGWGQPHGGGWGQGGGTHSQWNKPSKPKTNMKHMAGAAAAGA
VVGGLGVYMLGSAMISADKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCSVMHEALH
NHYTQKSLSLSPGK;
(SEQ ID NO. 26)
THSQWNKPSKPKTNMKHMAGAAAAGAVVGGLGVYMLGSAMISADKTHTC
PPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK;
(SEQ ID NO. 27)
PQGGGGWGQPHGGGWGQPHGGGWGQPHGGGWGQPHGGGWGQGGG
THSQWNKPSKPKTNMKHMAGAAAAGAVVGGLGGYMLGSAMISADKTHTCPPCPAPE
LLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK;
(SEQ ID NO. 28)
KKRPKPGGWNTGGSRYPGQGSPGGNRYPPQGGGGWGQPHGGGWGQP
HGGGWGQPHGGGWGQPHGGGWGQGGGTHSQWNKPSKPKTNMKHMAGAAAAGA
VVGGLGGYMLGSAMISADKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCSVMHEALH
NHYTQKSLSLSPGK;
(SEQ ID NO. 29)
THSQWNKPSKPKTNMKHMAGAAAAGAVVGGLGGYMLGSAMISADKTHTC
PPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK;
(SEQ ID NO. 30)
MANLGCWMLVLFVATWSDLGLCKKRPKPGGWNTGGSRYPGQGSPGGNR
YPPQGGGGWGQPHGGGWGQPHGGGWGQPHGGGWGQPHGGGWGQGGGTHSQ
WNKPSKPKTNMKHMAGAAAAGAVVGGLGVYMLGSAMSRPIIHFGSDYEDRYYRENM
HRYPNQVYYRPMDEYSNQNNFVHDCVNITIKQHTVTTTTKGENFTETDVKMMERVVE
QMCITQYERESQAYYQRGSSMVLFSSPPVILLISFLIFLIVGISADKTHTCPPCPAPELLG
GPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK;
(SEQ ID NO. 31)
MANLGCWMLVLFVATWSDLGLCKKRPKPGGWNTGGSRYPGQGSPGGNR
YPPQGGGGWGQPHGGGWGQPHGGGWGQPHGGGWGQPHGGGWGQGGGTHSQ
WNKPSKPKTNMKHMAGAAAAGAVVGGLGGYVLGSAMSRPIIHFGSDYEDRYYRENM
HRYPNQVYYRPMDEYSNQNNFVHDCVNITIKQHTVTTTTKGENFTETDVKMMERVVE
QMCITQYERESQAYYQRGSSMVLFSSPPVILLISFLIFLIVGISADKTHTCPPCPAPELLG
GPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK;
(SEQ ID NO. 32)
KKRPKPGGWNTGGSRYPGQGSPGGNRYPPQGGGGWGQPHGGGWGQP
HGGGWGQPHGGGWGQPHGGGWGQGGGTHSQWNKPSKPKTNMKHMAGAAAAGA
VVGGLGGYVLGSAMISADKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCSVMHEALH
NHYTQKSLSLSPGK;
(SEQ ID NO. 33)
PQGGGGWGQPHGGGWGQPHGGGWGQPHGGGWGQPHGGGWGQGGG
THSQWNKPSKPKTNMKHMAGAAAAGAVVGGLGGYVLGSAMISADKTHTCPPCPAPE
LLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK;
(SEQ ID NO. 34)
THSQWNKPSKPKTNMKHMAGAAAAGAVVGGLGGYVLGSAMISADKTHTC
PPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK;
and
(SEQ ID NO. 35)
MANLGCWMLVLFVATWSDLGLCKKRPKPGGWNTGGSRYPGQGSPGGNR
YPPQGGGGWGQPHGGGWGQPHGGGWGQPHGGGWGQPHGGGWGQGGGTHSQ
WNKPSKPKTNMKHMAGAAAAGAVVGGLGGYMLGSAMSRPIIHFGSDYEDRYYRENM
HRYPNQVYYRPMDEYSNQNNFVHDCVNITIKQHTVTTTTKGENFTETDVKMMERVVE
QMCITQYKRESQAYYQRGSSMVLFSSPPVILLISFLIFLIVGISADKTHTCPPCPAPELLG
GPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK;
(SEQ ID NO. 36)
MYRMQLLSCIALSLALVTNSISAMVRSMANLGCWMLVLFVATWSDLGLCKK
RPKPGGWNTGGSRYPGQGSPGGNRYPPQGGGGWGQPHGGGWGQPHGGGWGQ
PHGGGWGQPHGGGWGQGGGTHSQWNKPSKPKTNMKHMAGAAAAGAVVGGLGGY
MLGSAMSRPIIHFGSDYEDRYYRENMHRYPNQVYYRPMDEYSNQNNFVHDCVNITIK
QHTVTTTTKGENFTETDVKMMERVVEQMCITQYERESQAYYQRGSSMVLFSSPPVILL
ISFLIFLIVGISADKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQK
SLSLSPGK;
(SEQ ID NO. 37)
MYRMQLLSCIALSLALVTNSISAMVRSPQGGGGWGQPHGGGWGQPHGGG
WGQPHGGGWGQPHGGGWGQGGGTHSQWNKPSKPKTNMKHMAGAAAAGAVVGG
LGVYMLGSAMISADKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHNHYT
QKSLSLSPGK;
(SEQ ID NO. 38)
MYRMQLLSCIALSLALVTNSISAMVRSPQGGGGWGQPHGGGWGQPHGGG
WGQPHGGGWGQPHGGGWGQGGGTHSQWNKPSKPKTNMKHISADKTHTCPPCPA
PELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFF
LYSKLTVDKSRWQQGNVFSCSVMHEALHNHYTQKSLSLSPGK;
(SEQ ID NO. 39)
MYRMQLLSCIALSLALVTNSISAMVRSKKRPKPGGWNTGGSRYPGQGSPG
GNRYPPQGGGGWGQPHGGGWGQPHGGGWGQPHGGGWGQPHGGGWGQGGGT
HSQWNKPSKPKTNMKHISADKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHE
ALHNHYTQKSLSLSPGK;
(SEQ ID NO. 40)
MYRMQLLSCIALSLALVTNSISAMVRSKKRPKPGGWNTGGSRYPGQGSPG
GNRYPPQGGGGWGQPHGGGWGQPHGGGWGQPHGGGWGQPHGGGWGQGGGT
HSQWNKPSKPKTNMKHMAGAAAAGAVVGGLGVYMLGSAMISADKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK;
(SEQ ID NO. 41)
MYRMQLLSCIALSLALVTNSISAMVRSTHSQWNKPSKPKTNMKHMAGAAAA
GAVVGGLGVYMLGSAMISADKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHE
ALHNHYTQKSLSLSPGK;
(SEQ ID NO. 42)
MYRMQLLSCIALSLALVTNSISAMVRSPQGGGGWGQPHGGGWGQPHGGG
WGQPHGGGWGQPHGGGWGQGGGTHSQWNKPSKPKTNMKHMAGAAAAGAVVGG
LGGYMLGSAMISADKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHY
TQKSLSLSPGK;
(SEQ ID NO. 43)
MYRMQLLSCIALSLALVTNSISAMVRSKKRPKPGGWNTGGSRYPGQGSPG
GNRYPPQGGGGWGQPHGGGWGQPHGGGWGQPHGGGWGQPHGGGWGQGGGT
HSQWNKPSKPKTNMKHMAGAAAAGAVVGGLGGYMLGSAMISADKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK;
(SEQ ID NO. 44)
MYRMQLLSCIALSLALVTNSISAMVRSTHSQWNKPSKPKTNMKHMAGAAAA
GAVVGGLGGYMLGSAMISADKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHE
ALHNHYTQKSLSLSPGK;
(SEQ ID NO. 45)
MYRMQLLSCIALSLALVTNSISAMVRSMANLGCWMLVLFVATWSDLGLCKK
RPKPGGWNTGGSRYPGQGSPGGNRYPPQGGGGWGQPHGGGWGQPHGGGWGQ
PHGGGWGQPHGGGWGQGGGTHSQWNKPSKPKTNMKHMAGAAAAGAVVGGLGVY
MLGSAMSRPIIHFGSDYEDRYYRENMHRYPNQVYYRPMDEYSNQNNFVHDCVNITIK
QHTVTTTTKGENFTETDVKMMERVVEQMCITQYERESQAYYQRGSSMVLFSSPPVILL
ISFLIFLIVGISADKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQK
SLSLSPGK;
(SEQ ID NO. 46)
MYRMQLLSCIALSLALVTNSISAMVRSMANLGCWMLVLFVATWSDLGLCKK
RPKPGGWNTGGSRYPGQGSPGGNRYPPQGGGGWGQPHGGGWGQPHGGGWGQ
PHGGGWGQPHGGGWGQGGGTHSQWNKPSKPKTNMKHMAGAAAAGAVVGGLGGY
VLGSAMSRPIIHFGSDYEDRYYRENMHRYPNQVYYRPMDEYSNQNNFVHDCVNITIKQ
HTVTTTTKGENFTETDVKMMERVVEQMCITQYERESQAYYQRGSSMVLFSSPPVILLI
SFLIFLIVGISADKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKSL
SLSPGK;
(SEQ ID NO. 47)
MYRMQLLSCIALSLALVTNSISAMVRSKKRPKPGGWNTGGSRYPGQGSPG
GNRYPPQGGGGWGQPHGGGWGQPHGGGWGQPHGGGWGQPHGGGWGQGGGT
HSQWNKPSKPKTNMKHMAGAAAAGAVVGGLGGYVLGSAMISADKTHTCPPCPAPEL
LGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK;
(SEQ ID NO. 48)
MYRMQLLSCIALSLALVTNSISAMVRSPQGGGGWGQPHGGGWGQPHGGG
WGQPHGGGWGQPHGGGWGQGGGTHSQWNKPSKPKTNMKHMAGAAAAGAVVGG
LGGYVLGSAMISADKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHNHYT
QKSLSLSPGK;
(SEQ ID NO. 49)
MYRMQLLSCIALSLALVTNSISAMVRSTHSQWNKPSKPKTNMKHMAGAAAA
GAVVGGLGGYVLGSAMISADKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHE
ALHNHYTQKSLSLSPGK;
and
(SEQ ID NO. 50)
MYRMQLLSCIALSLALVTNSISAMVRSMANLGCWMLVLFVATWSDLGLCKK
RPKPGGWNTGGSRYPGQGSPGGNRYPPQGGGGWGQPHGGGWGQPHGGGWGQ
PHGGGWGQPHGGGWGQGGGTHSQWNKPSKPKTNMKHMAGAAAAGAVVGGLGGY
MLGSAMSRPIIHFGSDYEDRYYRENMHRYPNQVYYRPMDEYSNQNNFVHDCVNITIK
QHTVTTTTKGENFTETDVKMMERVVEQMCITQYKRESQAYYQRGSSMVLFSSPPVILL
ISFLIFLIVGISADKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQK
SLSLSPGK.

Prion Protein

Functions of Prion Proteins

The prion-Fc region fusion proteins disclosed herein include human prion proteins. It is known that the human prion protein is capable of binding to protein aggregates associated with neurodegenerative diseases. Accordingly, the human prion protein portion included in the prion-Fc region fusion protein functions to bind the prion-Fc region fusion protein to neurotoxic protein aggregates, thereby enabling it to produce its intended effect.

In one embodiment, the human prion protein can bind an alpha-synuclein aggregate, an amyloid beta aggregate, a tau aggregate, a TDP-43 aggregate, and/or a prion aggregate. In one example, the aggregates may be in oligomeric, protofibril, fibril, and/or Lewy body forms.

Human Prion Protein

The prion-Fc region fusion proteins disclosed herein are characterized in that they comprise prion proteins found in humans. It is known that prion proteins are found in other animals as well as humans and that the sequences of the prion proteins differ between species. For example, mouse prion protein (MoPrP) and human prion protein (hPrP) are classified as the same prion protein but have different amino acid sequences. Since the prion-Fc region fusion protein is intended for therapeutic use in humans, it must contain a prion protein sequence found in humans. If the prion protein is not a human prion protein found in humans, but a prion protein of a different species, 1) there is a possibility that the prion protein may not specifically bind to protein aggregates that are neurotoxic to humans, and 2) the prion protein is of “foreign origin” and may have adverse effects, such as an immune response when administered to the human body. Thus, the prion-Fc region fusion protein is characterized in that it comprises a human prion protein.

In one embodiment, the prion-Fc region fusion protein may comprise normal prion protein (PrPC) found in humans.

Example Human Prion Protein Sequence

In one embodiment, the human prion protein has the sequence

(SEQ ID NO. 1)
MANLGCWMLVLFVATWSDLGLCKKRPKPGGWNTGGSRYPGQGSPGGNRY
PPQGGGGWGQPHGGGWGQPHGGGWGQPHGGGWGQPHGGGWGQGGGTHSQ
WNKPSKPKTNMKHMAGAAAAGAVVGGLGGYMLGSAMSRPIIHFGSDYED
RYYRENMHRYPNQVYYRPMDEYSNQNNFVHDCVNITIKQHTVTTTTKGE
NFTETDVKMMERVVEQMCITQYERESQAYYQRGSSMVLFSSPPVILLIS
FLIFLIVG

Fragment of Human Prion Protein

The use of a prion protein in the prion-Fc region fusion protein is intended to utilize the ability of the prion protein to bind to neurotoxic protein aggregates. Accordingly, only the portion of the prion protein that serves to bind to the neurotoxic protein aggregates may be used. In one embodiment, the prion-Fc region fusion protein may comprise a fragment of a human prion protein. Specifically, the fragment of a human prion protein is a portion of the human prion protein that is capable of binding to a pathogenic agent that causes the neurodegenerative neurological disease.

Example Section of a Human Prion Protein

In one embodiment, the human prion protein may be a fragment comprising amino acids 51st through 111st of the human prion protein of SEQ ID NO. 1. In one embodiment, the human prion protein may be a fragment comprising amino acids 51st through 134th of the human prion protein of SEQ ID NO. 1.

In one embodiment, the human prion protein may be a fragment comprising amino acids 23rd to 111 st of the human prion protein of SEQ ID NO. 1.

In one embodiment, the human prion protein may be a fragment comprising amino acids 23rd to 134th of the human prion protein of SEQ ID NO. 1.

In one embodiment, the human prion protein may be a fragment comprising the 95th amino acid to 134th amino acid of the human prion protein of SEQ ID NO. 1.

Example of a Cleaved Sequence of a Human Prion Protein

In one embodiment, the fragment of human prion protein may be selected from the following.

(SEQ ID NO. 2)
PQGGGGWGQPHGGGWGQPHGGGWGQPHGGGWGQPHGGGWGQGGGTHSQW
NKPSKPKTNMKH,
(SEQ ID NO. 3)
PQGGGGWGQPHGGGWGQPHGGGWGQPHGGGWGQPHGGGWGQGGGTHSQW
NKPSKPKTNMKHMAGAAAAGAVVGGLGGYMLGSAM,
(SEQ ID NO. 4)
KKRPKPGGWNTGGSRYPGQGSPGGNRYPPQGGGGWGQPHGGGWGQPHGG
GWGQPHGGGWGQPHGGGWGQGGGTHSQWNKPSKPKTNMKH,
(SEQ ID NO. 5)
KKRPKPGGWNTGGSRYPGQGSPGGNRYPPQGGGGWGQPHGGGWGQPHGG
GWGQPHGGGWGQPHGGGWGQGGGTHSQWNKPSKPKTNMKHMAGAAAAGA
VVGGLGGYMLGSAM,
and
(SEQ ID NO. 6)
THSQWNKPSKPKTNMKHMAGAAAAGAVVGGLGGYMLGSAM

Variants of the human prion protein and its fragments

The prion protein may be a variant of a human prion protein, a fragment of a human prion protein variant, and/or a variant of a human prion protein fragment. The variant of the prion protein is a variant of all or part of the amino acid sequence of a prion protein found in nature. The variant of the prion protein may have certain functions or properties added, removed, and/or improved compared to the prion protein found in nature.

In one embodiment, the variants of prion proteins may have removed properties that make them toxic when accumulated in the body compared to prion proteins found in nature.

Examples of Variants of the Human Prion Protein and its Fragments

In one embodiment, the human prion protein and variants thereof may be a protein having a 127th G to V modification of the amino acid sequence of the human prion protein of SEQ ID NO: 1, or a fragment comprising the modified amino acid. In one embodiment, the human prion protein and a variant thereof may be a protein in which the 129th M of the amino acid sequence of the human prion protein of SEQ ID NO. 1 is modified to a V, or a fragment comprising the modified amino acid. In one embodiment, the human prion protein and variants thereof may be a protein in which the 219th E of the amino acid sequence of the human prion protein of SEQ ID NO.1 is modified to a K, or a fragment comprising the modified amino acid.

Example Sequences of Variants of the Human Prion Protein and its Fragments

One embodiment, a human prion protein and a variant of a fragment thereof may be selected from the following

(SEQ ID NO. 7)
MANLGCWMLVLFVATWSDLGLCKKRPKPGGWNTGGSRYPGQGSPGGNRY
PPQGGGGWGQPHGGGWGQPHGGGWGQPHGGGWGQPHGGGWGQGGGTHSQ
WNKPSKPKTNMKHMAGAAAAGAVVGGLGVYMLGSAMSRPIIHFGSDYED
RYYRENMHRYPNQVYYRPMDEYSNQNNFVHDCVNITIKQHTVTTTTKGE
NFTETDVKMMERVVEQMCITQYERESQAYYQRGSSMVLFSSPPVILLIS
FLIFLIVG,
(SEQ ID NO. 8)
PQGGGGWGQPHGGGWGQPHGGGWGQPHGGGWGQPHGGGWGQGGGTHSQW
NKPSKPKTNMKHMAGAAAAGAVVGGLGVYMLGSAM,
(SEQ ID NO. 9)
KKRPKPGGWNTGGSRYPGQGSPGGNRYPPQGGGGWGQPHGGGWGQPHGG
GWGQPHGGGWGQPHGGGWGQGGGTHSQWNKPSKPKTNMKHMAGAAAAGA
VVGGLGVYMLGSAM,
(SEQ ID NO. 10)
THSQWNKPSKPKTNMKHMAGAAAAGAVVGGLGVYMLGSAM,
(SEQ ID NO. 11)
MANLGCWMLVLFVATWSDLGLCKKRPKPGGWNTGGSRYPGQGSPGGNRY
PPQGGGGWGQPHGGGWGQPHGGGWGQPHGGGWGQPHGGGWGQGGGTHSQ
WNKPSKPKTNMKHMAGAAAAGAVVGGLGGYVLGSAMSRPIIHFGSDYED
RYYRENMHRYPNQVYYRPMDEYSNQNNFVHDCVNITIKQHTVTTTTKGE
NFTETDVKMMERVVEQMCITQYERESQAYYQRGSSMVLFSSPPVILLIS
FLIFLIVG,
(SEQ ID NO. 12)
KKRPKPGGWNTGGSRYPGQGSPGGNRYPPQGGGGWGQPHGGGWGQPHGG
GWGQPHGGGWGQPHGGGWGQGGGTHSQWNKPSKPKTNMKHMAGAAAAGA
VVGGLGGYVLGSAM,
(SEQ ID NO. 13)
PQGGGGWGQPHGGGWGQPHGGGWGQPHGGGWGQPHGGGWGQGGGTHSQW
NKPSKPKTNMKHMAGAAAAGAVVGGLGGYVLGSAM,
(SEQ ID NO. 14)
THSQWNKPSKPKTNMKHMAGAAAAGAVVGGLGGYVLGSAM,
and
(SEQ ID NO. 15)
MANLGCWMLVLFVATWSDLGLCKKRPKPGGWNTGGSRYPGQGSPGGNRY
PPQGGGGWGQPHGGGWGQPHGGGWGQPHGGGWGQPHGGGWGQGGGTHSQ
WNKPSKPKTNMKHMAGAAAAGAVVGGLGGYMLGSAMSRPIIHFGSDYED
RYYRENMHRYPNQVYYRPMDEYSNQNNFVHDCVNITIKQHTVTTTTKGE
NFTETDVKMMERVVEQMCITQYKRESQAYYQRGSSMVLFSSPPVILLIS
FLIFLIVG.

Fc Area

Functions of the Fc Region

The prion-Fc region fusion proteins disclosed herein comprise the Fc region of an immunoglobulin. The Fc region has the function of binding to Fc receptors on macrophages and/or microglia to induce phagocytosis.

Furthermore, the Fc region can be recycled by binding to FcRn (Fc Receptor neonate) on the cell surface, which in turn has the effect of increasing the half-life of the prion-Fc region fusion protein. In conclusion, the Fc region contained in the prion-Fc region fusion protein functions to 1) induce elimination of the target (neurotoxic protein aggregate) to which the fusion protein is bound, and 2) recirculate through FcRn to stably retain in the body.

Fc Region Example

The Fc regions included in the prion-Fc region fusion proteins disclosed herein are not substantially limited to achieving the aforementioned objectives.

In one embodiment, the Fc region may be the Fc region of a human immunoglobulin protein. Specifically, the Fc region may be the Fc region of human immunoglobulin G. More specifically, the Fc region may be an Fc region derived from human IgG1, IgG2, IgG3, and/or IgG4.

In another embodiment, the Fc region may be a variant of the Fc region of a human immunoglobulin protein. The variant of the Fc region may have one or more amino acids of its amino acid sequence removed, added, and/or substituted compared to the Fc region of a human immunoglobulin protein found in nature. Specifically, the Fc region may be a variant of an Fc region derived from human IgG1, IgG2, IgG3, and/or IgG4.

In another embodiment, the Fc region may be the Fc region of a mouse immunoglobulin protein. Specifically, the Fc region may be the Fc region of mouse immunoglobulin G. More specifically, the Fc region may be an Fc region derived from mouse IgG1, IgG2, IgG3, and/or IgG4.

In another embodiment, the Fc region may be a variant of the Fc region of a mouse immunoglobulin protein. The variant of the Fc region may have one or more amino acids of its amino acid sequence removed, added, and/or substituted compared to the Fc region of a mouse immunoglobulin protein found in nature. Specifically, the Fc region may be a variant of an Fc region derived from IgG1, IgG2, IgG3, and/or IgG4 of a mouse.

Example Fc Region Sequence

In one embodiment, the Fc region may be represented by the following sequence:

(SEQ ID NO. 16)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPGK;
and
(SEQ ID NO. 51)
PPCPSCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQE
GNVFSCSVMHEALHNHYTQKSLSLSLGK.

Other Configurations

Other Configuration Overview

The prion-Fc region fusion proteins disclosed herein may further comprise other constructs other than the prion protein and the Fc region. Such other constructs may be, for example, but not limited to, linker peptides for connecting the respective constructs, secretion signal peptides that have important functions when expressed intracellularly in vector form, and the like.

Secretory Signal Peptides

The prion-Fc region fusion protein may comprise a secretion signal peptide. The secretion signal peptide serves as a signal to cause the cell to secrete the prion-Fc region fusion protein out of the cell when the prion-Fc region fusion protein is to be expressed in the cell. The secretion signal peptide is not particularly limited to any peptide that functions as described above. In one embodiment, the secretion signal peptide may be a known secretion signal peptide. In one embodiment, the secretion signal peptide may be a secretion signal peptide of a protein secreted in the body. In one embodiment, the secretion signal peptide may be located at the N-terminal end of the prion-Fc region fusion protein. In another embodiment, the secretion signal peptide may be located at the C-terminal end of the prion-Fc region fusion protein.

Secretory Signal Peptide Example

In one embodiment, the secretion signal peptide may be represented by a sequence selected from MYRMQLLSCIALSLALVTNS(SEQ ID NO.17), MAFLWLLSCWALLGTTFG(SEQ ID NO.52), and MNLLLILTFVAAAVA(SEQ ID NO.53).

Linker

The prion-Fc region fusion protein may comprise a linker. The linker may be, but is not limited to, a peptide that functions to link the respective portions included in the prion-Fc region fusion protein.

In one embodiment, the human prion protein and the Fc region included in the prion-Fc region fusion protein may be linked via a linker. In one embodiment, the prion-Fc region fusion protein may be linked via a linker to the secretion signal peptide. In one embodiment, the linker may be a linker disclosed in the prior art Fusion Protein Linkers: Property, Design, and Functionality, Chen et al. 2013, Advanced Drug Delivery Reviews, Vol. 65, or a linker designed by a method disclosed in the prior art.

Linker Examples

In one embodiment, the linker may be represented by a sequence selected from the following:

ISA;
(SEQ ID NO. 18)
ISAMVRS;
(G)n;
(SEQ ID NO. 19)
(GGGGS)n;
(SEQ ID NO. 20)
(EAAAK)n;
and
(XP)n,

where n is an integer greater than or equal to 1 and X is any standard amino acid.

Vectors for Expressing Prion-Fc Region Fusion Protein

Overview of Vectors to Express Prion-Fc Region Fusion Protein

Disclosed herein are vectors capable of expressing the prion-Fc region fusion proteins. While the prion-Fc region fusion protein may be externally produced and administered to a subject, the vector capable of expressing the prion-Fc region fusion protein may be administered to the subject's body to cause the subject's cells to produce and secrete the prion-Fc region fusion protein themselves. A vector capable of expressing the prion-Fc region fusion protein comprises a nucleic acid encoding the prion-Fc region fusion protein, and additional constructs for expressing the prion-Fc region fusion protein, such as a promoter sequence.

Nucleic Acid Encoding a Prion-Fc Region Fusion Protein

A vector capable of expressing the prion-Fc region fusion protein comprises a nucleic acid encoding a prion-Fc region fusion protein. Wherein the prion-Fc region fusion protein is as described above.

In one embodiment, the nucleic acid encoding the prion-Fc region fusion protein may be the prion-Fc region fusion protein disclosed in the paragraph “Example prion-Fc region fusion protein sequences”. In one embodiment, the nucleic acid encoding the prion-Fc region fusion protein may be a protein having a secretion signal peptide linked to the C-terminus or N-terminus of the prion-Fc region fusion protein disclosed in the paragraph “Example prion-Fc region fusion protein sequences”.

Example Nucleic Acid Sequence Encoding a Prion-Fc Region Fusion Protein

In one embodiment, the nucleic acid sequence encoding the prion-Fc region fusion protein may comprise the following sequence:

Configuring Vector Additions

A vector capable of expressing the prion-Fc region fusion protein further comprises a nucleic acid encoding the prion-Fc region fusion protein that enables the nucleic acid to be translated into a protein in a target cell. The additional constructs may include, for example, but are not limited to, a regulatory/control component, a promoter, and a transcription termination signal.

In one embodiment, a vector capable of expressing the prion-Fc region fusion protein comprises a promoter. In one example, the promoter may be a cell type specific promoter. Specifically, the promoter may be a neuronal, epithelial cell, or ependymal cell specific promoter. In another example, the promoter may be a promoter having a specific function. Specifically, the promoter may be a drug-inducible promoter, an inflammation-inducible promoter, or an inflammation-inducible promoter.

Nucleic Acid Type

Nucleic acids encoding the prion-Fc region fusion proteins, and/or further constitutive nucleic acids for expressing the fusion proteins, are not particularly limited in type, provided that they are capable of achieving the aforementioned purposes in a cell. In one embodiment, the nucleic acid may be selected from DNA, mRNA, and chemically modified nucleic acids.

Vector Form 1—Common Forms

Vectors capable of expressing the prion-Fc region fusion proteins include, but are not particularly limited to, those capable of expressing the prion-Fc region fusion proteins comprising the constructs described above. In one embodiment, the vector capable of expressing the prion-Fc region fusion protein can be in the form selected from plasmids, phages, naked DNA, DNA complexes, mRNA, and viral vectors. Here, the viral vector may be selected from retroviruses, lentiviruses, adenoviruses, adeno-associated viruses, vaccinia viruses, fox viruses, and herpes simplex viruses.

Vector Form 2—Adeno-Associated Viruses

In one embodiment, the vector capable of expressing the prion-Fc region fusion protein may be in the form of an adeno-associated virus. In one embodiment, the adeno-associated virus may be a serotype modified to be blood-brain barrier permeable. In one embodiment, the adeno-associated virus may be a serotype modified to be able to transfect a target cell specifically. Specifically, the target cell may be selected from a neuron, an epithelial cell, and an ependymal cell. In one embodiment, the adeno-associated virus may have its capsid surface engineered to better infect the Central Nervous System (CNS). In one embodiment, the adeno-associated virus may be a serotype selected from the group consisting of AAV1, AAV2, AAV4, AAV5, AAV8, and AAV9.

A Composition Comprising a Prion-Fc Region Fusion Protein or a Vector Capable of Expressing the Same

Compositions Comprising a Prion-Fc Region Fusion Protein or a Vector Capable of Expressing the Same

Disclosed herein are compositions comprising a prion-Fc region fusion protein, or a vector capable of expressing the same. The compositions are suitably formulated, formulated with, and/or containing additional materials for use with the prion-Fc region fusion protein, or vector capable of expressing the same, for various purposes.

Dosage Form of the Composition

In one embodiment, the composition comprising the prion-Fc region fusion protein, or a vector capable of expressing the same, may be formulated for oral administration, injectable administration, mucosal administration, transdermal administration, and/or topical skin administration. In one embodiment, a composition comprising a prion-Fc region fusion protein, or a vector capable of expressing the same, may be formulated for use as an injectable. Specifically, the composition may be formulated for cerebrovascular injection.

Configuration for Preparing Prion-Fc Domain Fusion Protein

In one embodiment, the prion-Fc region fusion protein can be formulated as troches, lozenges, tablets, aqueous suspensions, oily suspensions, crude powders, granules, emulsions, hard capsules, soft capsules, syrups, or elixirs. In one embodiment, to formulate the prion-Fc region fusion protein for oral administration, a binder such as lactose, saccharose, sorbitol, mannitol, starch, amylopectin, cellulose or gelatin; an excipient such as dicalcium phosphate; disintegrating agents such as corn starch or sweet potato starch; lubricants such as magnesium stearate, calcium stearate, sodium stearyl fumarate, or polyethylene glycol wax; sweeteners, flavors, syrups, and the like. Furthermore, in the case of an encapsulant, in addition to the above-mentioned substances, a liquid carrier such as a fatty oil can be used. In one embodiment, the prion-Fc region fusion protein can be formulated as an injection, suppository, powder for respiratory inhalation, aerosol for spray, ointment, powder for application, oil, or cream. In one embodiment, to formulate the prion-Fc region fusion protein for parenteral administration, a sterile aqueous solution, non-aqueous solvent, suspension, emulsion, lyophilized preparation, topical preparation, or the like can be used, wherein the non-aqueous solvent or suspension can be propylene glycol, polyethylene glycol, vegetable oil such as olive oil, injectable esters such as ethyl oleate, or the like. In one embodiment, to formulate the prion-Fc region fusion protein into an injectable solution, the prion-Fc region fusion protein may be mixed in water with a stabilizer or buffer to form a solution or suspension, which may be formulated for unit dosing in ampoules or vials. In one embodiment, the prion-Fc region fusion protein can be formulated as an aerosol by mixing the prion-Fc region fusion protein with an additive, such as a propellant, to produce a water-dispersible concentrate or a wettable powder. In one embodiment, when the prion-Fc region fusion protein is formulated for transdermal use, animal oil, vegetable oil, wax, paraffin, starch, tricanth, cellulose derivatives, polyethylene glycol, silicone, bentonite, silica, talc, zinc oxide, and the like can be added to the prion-Fc region fusion protein as a carrier to produce an ointment, cream, powder for application, oil, topical agent, and the like.

Constructs for Preparing a Vector Capable of Expressing a Prion-Fc Domain Fusion Protein

Vectors capable of expressing the prion-Fc region fusion proteins may be prepared by methods known to those of ordinary skill in the art.

In one embodiment, a vector capable of expressing the formulated prion-Fc region fusion protein may comprise one or more of the following selected from the group consisting of: Naked nucleic acid; Cationic peptide-conjugated nucleic acid (Protamine); Positively charged oil-water cationic nanoemulsion; Nucleic acid conjugated to a chemically modified dendrimer and complexed with polyethylene glycol and PEG-lipid (Modified dendrimer nanoparticle); Nucleic acids complexed with protamine in PEG-lipid nanoparticles (Protamine liposome); Nucleic acids complexed with cationic polymers such as polyethylenimine, PEI (Cationic polymer); Nucleic acids complexed with cationic polymers such as PEI and lipid components (Cationic polymer liposome); Nucleic acids complexed with polysaccharide polymers such as chitosan (Polysaccharide particle); Nucleic acids complexed with cationic lipid nanoparticle polymers (Cationic lipid nanoparticle); Nucleic acid complexed with cationic lipid and cholesterol (Cationic lipid-cholesterol nanoparticle); and Nucleic acid complexed with cationic lipid and cholesterol and PEG-lipid (Cationic lipid-cholesterol-PEG nanoparticle).

In one embodiment, a vector capable of expressing the formulated prion-Fc region fusion protein may comprise lipid nanoparticles (LNPs). In the embodiments, the lipid nanoparticles may be ionizable cationic lipids, phospholipids, cholesterol, and/or lipid-anchored polyethylene glycol. Specifically, the ionizable cationic lipid may be one or more selected from the following: DLin-DMA; DLin-KC2-DMA; DLin-MC3-DMA; C12-200; cKK-E12; DLin-MC3-DMA derivative L319 (Alnylam and AlCana Technologies); C12-200 and cKK-E12 derivatives (Anderson group); COVID-19 vaccine lipids ALC-0315 and SM-102; TT3 and its biodegradable derivative FTT5 (Dong's group); vitamin-derived lipids ssPalmE and VcLNP; A9 (Acuitas); L5 (Moderna); A18 Lipid; ATX Lipid (LUNAR® composition; Arcturus); and LP01 (Intellia Therapeutics). Specifically, the phospholipids may be one or more selected from the group consisting of 1,2-dioleoyl-sn-glycero-3-phosphoethanolamine (DOPE); and 1,2-distearoyl-sn-glycero-3-phosphocholine (DSPC).

In one embodiment, a vector capable of expressing the formulated prion-Fc region fusion protein may comprise a polymer-based delivery system. In the embodiment, the polymer-based delivery system may comprise one or more selected from the following: Polyethylenimine (PEI); polyamidoamine (PAMAM); polypropylenimine (PPI); and the polymer-based dendrimer.

In one embodiment, a vector capable of expressing the formulated prion-Fc region fusion protein may comprise a peptide-based delivery system. In the embodiments, the peptide-based delivery system may comprise protamine. Specifically, the formulated encoding nucleic acid may be a protamine-mRNA complex.

In one embodiment, a vector capable of expressing the formulated prion-Fc region fusion protein may comprise a cationic lipid-constituted liposome, lipoplex, and/or cationic emulsion (CNE). In the embodiments, the cationic lipid may be DOTMA (1,2-di-O-octadecenyl-3-trimethylammonium propane) and/or DOTAP (1,2-dioleoyl3-trimethylammonium-propane).

Mechanism of Action of Prion-Fc Region Fusion Protein

It is known that neurotoxic protein aggregates accumulate in the body and can be transmitted to other cells, thereby exacerbating neurodegenerative diseases. The prion protein portion of the prion-Fc region fusion protein provided herein can specifically bind to the neurotoxic protein aggregates, and the Fc region portion of the prion-Fc region fusion protein can bind to the Fc receptor of macrophages and/or microglia to induce phagocytosis. Thus, when the prion-Fc region fusion protein contact with the neurotoxic protein aggregates, i) the prion protein portion binds to the neurotoxic protein aggregates and prevents them from spreading to other cells, and ii) the Fc region portion binds to Fc receptors on macrophages and/or microglia to induce phagocytosis, resulting in the removal of the neurotoxic protein aggregates. Through the above mechanism of action, the prion-Fc region fusion protein can prevent the accumulation and dissemination of neurotoxic protein aggregates to other cells, thereby preventing, treating, and/or ameliorating diseases associated therewith.

Therapeutic uses of a composition comprising a prion-Fc region fusion protein or a vector capable of expressing the same

Outline Therapeutic Uses of Compositions Comprising Prion-Fc Region Fusion Proteins or Vectors Capable of Expressing the Same

Disclosed herein are therapeutic uses of compositions comprising the prion-Fc region fusion proteins or vectors capable of expressing the same. The compositions may be used for the treatment of neurodegenerative diseases, and appropriate routes of administration, dosages, and frequency of administration may be selected by one of ordinary skill in the art using methods known in the art to achieve the therapeutic uses.

Therapeutic Use of Compositions Comprising Prion-Fc Region Fusion Proteins or Vectors Capable of Expressing them 1—Targeted Substances

In one embodiment, a composition comprising the prion-Fc region fusion protein or a vector capable of expressing the same can be used to remove neurotoxic protein aggregates. Specifically, the neurotoxic protein aggregates may be alpha-synuclein aggregates, amyloid beta aggregates, tau aggregates, TDP-43 aggregates, and/or prion aggregates.

Therapeutic Use of Compositions Comprising Prion-Fc Region Fusion Proteins or Vectors Capable of Expressing them 2—Indications

In one embodiment, a composition comprising the prion-Fc region fusion protein or a vector capable of expressing the same can be used for the treatment of neurodegenerative disease. Specifically, the neurodegenerative diseases include Alzheimer's disease, Parkinson's disease, Lewy bodies disease, Pick's disease, Traumatic encephalopathy, Amyotrophic Lateral Sclerosis (ALS), Tauopathy; and Frontotemporal dementia.

Therapeutic Use of a Composition Comprising a Prion-Fc Region Fusion Protein or a Vector Capable of Expressing the Same 3—Method of Administration

In one embodiment, a composition comprising a prion-Fc region fusion protein or a vector capable of expressing the same can be administered to the brain of a subject. In one embodiment, the composition comprising the prion-Fc region fusion protein or vector capable of expressing the same can be administered to the subject by a method selected from the intracerebral injection, and intracerebroventricular injection (ICV). In one embodiment, a composition comprising a prion-Fc region fusion protein or a vector capable of expressing the same can be administered to a site selected from the Substantia Nigra, Cerebral Ventricle, and Striatum of a subject.

Therapeutic Uses of Compositions Comprising Prion-Fc Region Fusion Proteins or Vectors Capable of Expressing Them 4—Dosages

In one embodiment, a composition comprising the prion-Fc region fusion protein or a vector capable of expressing the same can be administered at a dose of from about 1 unit/kg to about 999 units/kg, based on the body weight of the subject. In one embodiment, the administered dose may be any numerical range included in the numerical range of the immediately preceding sentence. For example, the dosage may be from about 5 units/kg to about 20 units/kg. Here, the unit is selected from g, mg, μg, and ng.

Therapeutic Uses of Compositions Comprising Prion-Fc Region Fusion Proteins or Vectors Capable of Expressing Them 5—Frequency of Administration

In one embodiment, a composition comprising a prion-Fc region fusion protein or a vector capable of expressing the same can be administered to a subject once daily, or at least twice daily. In one embodiment, the composition comprising the prion-Fc region fusion protein or a vector capable of expressing the same can be administered to the subject at intervals of about 1 period to about 100 periods. In one embodiment, the dosing interval can be any numerical range included in the numerical range of the immediately preceding sentence. For example, the dosing interval may be from about 5 periods to about 20 periods. Here, the period is selected from the group consisting of minutes, hours, days, weeks, months, and years.

### Possible Embodiments of the Invention

Prion-Fc Region Fusion Protein 1

### Embodiment 1, Fusion Protein, PrP-Fc

Prion-Fc region fusion proteins, including:

Human prion protein; and

Immunoglobulin Fc region,

Wherein the C-terminus of the human prion protein and the N-terminus of the immunoglobulin Fc region are linked.

### Embodiment 2, Fusion Protein, Fc-PrP

Prion-Fc region fusion proteins, including:

Human prion protein; and

Immunoglobulin Fc region,

Wherein the N-terminus of the human prion protein and the C-terminus of the immunoglobulin Fc region are linked.

### Embodiment 3, Prion Protein Specific, Wild-Type Human Prion Protein

The human prion protein of any one of embodiments 1 to 2, wherein the human prion protein is a fusion protein that is all or part of a human prion protein found in nature.

### Embodiment 4, Limiting Wild-Type Human Prion Protein Sequence

The human prion protein of embodiment 3, wherein the human prion protein is a fusion protein that is all or part of the protein represented by SEQ ID NO: 1.

### Embodiment 5, Wild-Type Human Prion Protein Fragment Sequence Limited

The human prion protein of embodiment 4, wherein the human prion protein is a fusion protein represented by SEQ ID NO: 1 to SEQ ID NO: 6.

### Embodiment 6, Prion Protein Specific, Variant of Human Prion Protein

The fusion protein of any one of embodiments 1 to 2, wherein the human prion protein is a variant in which one or more amino acids of the amino acid sequence of the human prion protein found in nature have been added, altered, substituted, and/or removed.

### Embodiment 7, Variants of Human Prion Protein Limited

The fusion protein of embodiment 6, wherein the human prion protein is a fusion protein characterized in that at least one amino acid selected from the 127th amino acid, 129th amino acid, and 219th amino acid of the protein represented by SEQ ID NO: 1 is substituted for another.

### Embodiment 8,G127V

The fusion protein of embodiment 7, wherein the human prion protein is all or part of a protein in which the 127th amino acid of the protein represented by SEQ ID NO: 1 is substituted with a valine.

### Embodiment 9, a Fragment of G127V Variant

The human prion protein of embodiment 8, wherein the human prion protein is a fusion protein selected from SEQ ID NO: 7 to SEQ ID NO: 10.

### Embodiment 10, M129V

The fusion protein of embodiment 6, wherein the human prion protein is all or part of a protein in which the 129th amino acid of the protein represented by SEQ ID NO: 1 is substituted with a valine.

### Embodiment 11, a Fragment of M129V Variant

The human prion protein of embodiment 10, wherein the human prion protein is a fusion protein selected from SEQ ID NO: 11 to SEQ ID NO: 14.

### Embodiment 12, E219K

The fusion protein of embodiment 6, wherein the human prion protein is all or part of a protein in which the 219th amino acid of the protein represented by SEQ ID NO: 1 is substituted with Lysine.

### Embodiment 13, Immunoglobulin Fc Region-Derived Restriction

The fusion protein of any one of embodiments 1 to 12, wherein the immunoglobulin Fc region is a mouse or human immunoglobulin Fc region.

### Embodiment 14, Human Immunoglobulin Fc Region Specific

The fusion protein of embodiment 13, wherein the immunoglobulin Fc region is the Fc region of an immunoglobulin selected from mouse or human IgG1, IgG2, IgG3, and IgG4.

### Embodiment 15, Limiting Fc Sequence

The fusion protein of embodiment 13, wherein the immunoglobulin Fc region is represented by a sequence selected from the group consisting of:

(SEQ ID NO. 16)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPGK;
and
(SEQ ID NO. 51)
PPCPSCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQE
GNVFSCSVMHEALHNHYTQKSLSLSLGK.

### Embodiment 16, Add a Linker

The fusion protein of any one of embodiments 1 to 15, wherein the fusion protein further comprises a linker, and wherein the human prion protein and the immunoglobulin Fc region are linked via the linker.

### Embodiment 17, Linker Sequence Limited

The fusion protein of embodiment 16, wherein the linker is represented by a sequence selected from the group consisting of:

ISA;
(SEQ ID NO. 18)
ISAMVRS;
(G)n;
(SEQ ID NO. 19)
(GGGGS)n;
(SEQ ID NO. 20)
(EAAAK)n;
and
(XP)n,

### Embodiment 18, Add Secretion Signal Peptide, SS—PrP-Fc

The fusion protein of any one of embodiments 1 to 17, wherein the fusion protein further comprises a secretion signal peptide, wherein the C-terminus of the secretion signal peptide and the N-terminus of the human prion protein are linked.

### Embodiment 19, Adding a Secretion Signal Peptide and Linker

The fusion protein of any one of embodiments 1 to 15, wherein the fusion protein further comprises a secretion signal peptide, a first linker, and a second linker,

Wherein the C-terminus of the secretion signal peptide and the N-terminus of the human prion protein are linked via the first linker,

A fusion protein is characterized in that the C-terminus of the human prion protein and the N-terminus of the immunoglobulin Fc region are linked via the second linker.

### Embodiment 20, Secretion Signal Peptide Added, SS, N-Terminal

The fusion protein of any one of embodiments 1 to 17, wherein the fusion protein further comprises a secretion signal peptide, the secretion signal peptide is located at the N-terminal end of the amino acid sequence of the fusion protein.

### Embodiment 21, Secretion Signal Peptide Added, SS, C-Terminal

The fusion protein of any one of embodiments 1 to 17, wherein the fusion protein further comprises a secretion signal peptide, the secretion signal peptide is located at the C-terminal end of the amino acid sequence of the fusion protein.

### Embodiment 22, Linker Sequence Limited

The fusion protein of embodiment 19, wherein the first linker and the second linker are each independently represented by a sequence selected from the group consisting of:

ISA;
(SEQ ID NO. 18)
ISAMVRS;
(G)n;
(SEQ ID NO. 19)
(GGGGS)n;
(SEQ ID NO. 20)
(EAAAK)n;
and
(XP)n,

### Embodiment 23, Add a Use

The fusion protein of any one of embodiments 1 to 22, wherein the fusion protein is capable of inducing clearance of neurotoxic protein aggregates.

### Prion-Fc Region Fusion Protein 2

### Embodiment 24, Fusion Protein, Structural Formula

A prion-Fc region fusion protein represented by the following [Formula 1]:

hPrP−L1−Fc [Formula 1]

Where S is a secreted signal peptide, or absent, L1 is the first linker, or nonexistent,

hPrP is a human prion protein, a fragment of a human prion protein, a human prion protein variant, or a fragment of a human prion protein variant,

L2 is the second linker, or nonexistent,

Fc is the crystallizable region of a human or mouse immunoglobulin.

### Embodiment 25, Fusion Protein, Makushi

In Embodiment 24,

The secretion signal peptide is selected from the group consisting of SEQ ID NO: 17, SEQ ID NO: 52, or SEQ ID NO: 53,

The first linker is selected from the group consisting of ISA, ISAMVRS (SEQ ID NO. 18), (G)n, (GGGGS)n (SEQ ID NO. 19), (EAAAK)n (SEQ ID NO. 20), and (XP)n,

The hPrP is selected from the group consisting of SEQ ID NO. 1 to SEQ ID NO. 15,

The second linker is selected from ISA, ISAMVRS (SEQ ID NO. 18), (G)n, (GGGGS)n (SEQ ID NO. 19), (EAAAK)n (SEQ ID NO. 20), and (XP)n,

The Fc is a fusion protein selected from the group consisting of SEQ ID NO: 16 and SEQ ID NO: 51.

### Vectors for Expressing Prion-Fc Region Fusion Proteins

### Embodiment 26, Prion-Fc Region Encoding Nucleic Acid

A nucleic acid encodes a fusion protein of any one of embodiments 1 to 25.

### Embodiment 27, Nucleic Acid Specific

The nucleic acid of embodiment 26, wherein the nucleic acid is selected from DNA, mRNA, and chemically modified nucleic acids.

### Embodiment 28, a Vector Comprising a Prion-Fc Region Encoding Nucleic Acid

A vector capable of expressing a prion-Fc region fusion protein that includes:

The prion-Fc region encoding nucleic acid of embodiment 26; and Promoter.

### Embodiment 29, Non-Viral Vector

The vector of embodiment 28, wherein the vector is in the form selected from plasmid, phage, naked DNA, DNA complex, and mRNA.

### Embodiment 30, Viral Vector

The vector of embodiment 30, wherein the vector is in the form of a viral vector.

### Embodiment 31, Viral Vector Example

The viral vector of embodiment 30, wherein the viral vector is selected from the group consisting of a retrovirus, a lentivirus, an adenovirus, an adeno-associated virus, a vaccinia virus, a fox virus, and a herpes simplex virus.

### Embodiment 32, Adeno-Associated Viral Vector

The viral vector of embodiment 31, wherein the viral vector is an adeno-associated virus.

### Embodiment 33, Adeno-Associated Virus Sero Type

The vector of embodiment 32, wherein the the adeno-associated virus is a serotype selected from AAV1, AAV2, AAV4, AAV5, AAV8, and AAV9.

### Embodiment 34, Adeno-Associated Virus Capsid Modification

The vector of embodiment 32, wherein the adeno-associated virus is characterized in that the capsid surface thereof is engineered to allow the virus to better infect the Central Nervous System (CNS).

### A Composition Comprising a Prion-Fc Region Fusion Protein or a Vector Capable of Expressing the Same

### Embodiment 35, Composition

A composition that includes:

A vector capable of expressing the prion-Fc region fusion protein of any one of embodiments 1 to 25 or the prion-Fc region fusion protein of any one of embodiments 28 to 34; and

A pharmaceutically acceptable carrier.

### Embodiment 36, Fusion Protein Included, Carrier Specific

In embodiment 35, the composition comprising the prion-Fc region fusion protein of any one of embodiments 1 to 25, wherein the pharmaceutically acceptable carrier is at least one composition selected from the group consisting of:

Troches; lozenges; tablets; aqueous suspensions; oily suspensions; crude powders; granules; emulsions; hard capsules; soft capsules; syrups; elixirs; binders such as lactose, saccharose, sorbitol, mannitol, starch, amylopectin, cellulose or gelatin, etc; excipients, such as dicalcium phosphate; disintegrants, such as corn starch or sweet potato starch; lubricants, such as magnesium stearate, calcium stearate, sodium stearyl fumarate, or polyethylene glycol wax; Sweeteners; fragrances; syrups; non-aqueous solvents or suspensions, such as sterile aqueous solutions, propylene glycol, polyethylene glycol, vegetable oils such as olive oil; injectable esters such as ethylolates; emulsions; lyophilized preparations; topical agents; stabilizers; buffers; propellants; additives; animal oils; vegetable oils; waxes; paraffins; starches; tractants; cellulose derivatives; polyethylene glycol; silicones; bentonite; silica; talc; zinc oxide; and suitable mixtures thereof.

### Embodiment 37, Expression Vector Included, Carrier Specified

In embodiment 35, the composition comprises a vector capable of expressing the prion-Fc region fusion protein of any one of embodiments 28 to 34, wherein the pharmaceutically acceptable carrier is one or more compositions selected from the group consisting of:

Naked nucleic acid; cationic peptide-conjugated nucleic acid (Protamine); positively charged oil-water cationic nanoemulsion (Cationic nanoemulsion); nucleic acid conjugated to chemically modified dendrimers and complexed with polyethylene glycol and PEG-lipids (Modified dendrimer nanoparticle); Nucleic acids complexed with protamine in PEG-lipid nanoparticles (Protamine liposome); Nucleic acids complexed with cationic polymers such as polyethylenimine, PEI (Cationic polymer); Nucleic acids complexed with cationic polymers such as PEI and lipid components (Cationic polymer liposome); Nucleic acids complexed with polysaccharide polymers such as chitosan (Polysaccharide particles); Nucleic acids complexed with cationic lipid nanoparticle polymers (Cationic lipid nanoparticles); Nucleic acids complexed with cationic lipids and cholesterol (Cationic lipid-cholesterol nanoparticles); and cationic lipids and nucleic acids complexed with cholesterol and PEG-lipids (Cationic lipid-cholesterol-PEG nanoparticle); lipid nanoparticles (LNPs); ionizable cationic lipids (cationic lipids); phospholipids; and cholesterol; lipid-anchored polyethylene glycol; DLin-DMA; DLin-KC2-DMA; DLin-MC3-DMA; C12-200; cKK-E12; DLin-MC3-DMA derivative L319 (Alnylam and AlCana Technologies); C12-200 and cKK-E12 derivatives (Anderson Group); COVID-19 vaccine lipids ALC-0315 and SM-102; TT3 and its biodegradable derivative FTT5 (Dong's group); vitamin-derived lipids ssPalmE and VcLNP; A9 (Acuitas); L5 (Moderna); A18 Lipid; ATX Lipid (LUNAR® Compositions; Arcturus); and LP01 (Intellia Therapeutics); 1,2-dioleoyl-sn-glycero-3-phosphoethanolamine (DOPE); 1,2—distearoyl-sn-glycero-3-phosphocholine (DSPC); polymer-based delivery system; polyethylenimine (PEI); polyamidoamine (PAMAM); polypropylenimine; a dendrimer based on any of the foregoing polymers; a peptide-based delivery system; protamine; protamine-mRNA complexes; cationic lipid-constituted liposomes (liposomes); lipoplexes; cationic emulsions (CNEs); DOTMA (1,2-di-O-octadecenyl-3-trimethylammonium propane); DOTAP (1,2—dioleoyl3-trimethylammonium-propane); and suitable mixtures thereof.

### A Pharmaceutical Composition Comprising a Prion-Fc Region Fusion Protein or a Vector Capable of Expressing the Same

### Embodiment 38, Pharmaceutical Composition

A pharmaceutical composition for treating a neurodegenerative disease, comprising:

A vector capable of expressing the prion-Fc region fusion protein of any one of embodiments 1 to 25 or the prion-Fc region fusion protein of any one of embodiments 28 to 34; and

A pharmaceutically acceptable carrier.

### Embodiment 39, Fusion Protein Included, Carrier Specific

The pharmaceutical composition of embodiment 38, wherein the pharmaceutical composition comprises the prion-Fc region fusion protein of any one of embodiments 1 to 25 and the pharmaceutically acceptable carrier is at least one selected from the group consisting of:

Troches; lozenges; tablets; aqueous suspensions; oily suspensions; crude powders; granules; emulsions; hard capsules; soft capsules; syrups; elixirs; binders such as lactose, saccharose, sorbitol, mannitol, starch, amylopectin, cellulose or gelatin, etc; excipients, such as dicalcium phosphate; disintegrants, such as corn starch or sweet potato starch; lubricants, such as magnesium stearate, calcium stearate, sodium stearyl fumarate, or polyethylene glycol wax; Sweeteners; fragrances; syrups; non-aqueous solvents or suspensions, such as sterile aqueous solutions, propylene glycol, polyethylene glycol, vegetable oils such as olive oil; injectable esters such as ethylolates; emulsions; lyophilized preparations; topical agents; stabilizers; buffers; propellants; additives; animal oils; vegetable oils; waxes; paraffins; starches; tractants; cellulose derivatives; polyethylene glycol; silicones; bentonite; silica; talc; zinc oxide; and suitable mixtures thereof.

### Embodiment 40, Vector Included, Carrier Only

In embodiment 38, the composition comprises a vector capable of expressing the prion-Fc region fusion protein of any one of embodiments 28 to 34, wherein the pharmaceutically acceptable carrier is a pharmaceutical composition selected from the group consisting of one or more of the following:

Naked nucleic acid; cationic peptide-conjugated nucleic acid (Protamine); positively charged oil-water cationic nanoemulsion (Cationic nanoemulsion); nucleic acid conjugated to chemically modified dendrimers and complexed with polyethylene glycol and PEG-lipids (Modified dendrimer nanoparticle); Nucleic acids complexed with protamine in PEG-lipid nanoparticles (Protamine liposome); Nucleic acids complexed with cationic polymers such as polyethylenimine, PEI (Cationic polymer); Nucleic acids complexed with cationic polymers such as PEI and lipid components (Cationic polymer liposome); Nucleic acids complexed with polysaccharide polymers such as chitosan (Polysaccharide particles); Nucleic acids complexed with cationic lipid nanoparticle polymers (Cationic lipid nanoparticles); Nucleic acids complexed with cationic lipids and cholesterol (Cationic lipid-cholesterol nanoparticles); and cationic lipids and nucleic acids complexed with cholesterol and PEG-lipids (Cationic lipid-cholesterol-PEG nanoparticle); lipid nanoparticles (LNPs); ionizable cationic lipids (cationic lipids); phospholipids; and cholesterol; lipid-anchored polyethylene glycol; DLin-DMA; DLin-KC2-DMA; DLin-MC3-DMA; C12-200; cKK-E12; DLin-MC3-DMA derivative L319 (Alnylam and AlCana Technologies); C12-200 and cKK-E12 derivatives (Anderson Group); COVID-19 vaccine lipids ALC-0315 and SM-102; TT3 and its biodegradable derivative FTT5 (Dong's group); vitamin-derived lipids ssPalmE and VcLNP; A9 (Acuitas); L5 (Moderna); A18 Lipid; ATX Lipid (LUNAR® Compositions; Arcturus); and LP01 (Intellia Therapeutics); 1,2-dioleoyl-sn-glycero-3-phosphoethanolamine (DOPE); 1,2—distearoyl-sn-glycero-3-phosphocholine (DSPC); polymer-based delivery system; polyethylenimine (PEI); polyamidoamine (PAMAM); polypropylenimine; a dendrimer based on any of the foregoing polymers; a peptide-based delivery system; protamine; protamine-mRNA complexes; cationic lipid-constituted liposomes (liposomes); lipoplexes; cationic emulsions (CNEs); DOTMA (1,2-di-O-octadecenyl-3-trimethylammonium propane); DOTAP (1,2—dioleoyl3-trimethylammonium-propane); and suitable mixtures thereof.

### How to Remove Neurotoxic Protein Aggregates

### Embodiment 41, Method for Removing Neurotoxic Protein Aggregates

Methods for removing neurotoxic protein aggregates, comprising: inducing contact of the composition of any one of embodiments 35 to 37, and a neurotoxic protein aggregate present in the central nervous system of a subject.

### Embodiment 42, Neurotoxic Protein Aggregates Only

The method of embodiment 41, wherein the neurotoxic protein aggregates are at least one selected from the group consisting of alpha-synuclein aggregates, amyloid beta aggregates, tau aggregates, TDP-43 aggregates, and prion aggregates.

### Embodiment 43, Agglomerate Shape Only

The method of any one of embodiments 41 to 42, wherein the protein aggregates are in the form selected from oligomer, protofibril, mature fibril, and Lewy body.

### Embodiment 44, Targeted Central Nervous System Locations

The method of any one of embodiments 41 to 42, wherein the central nervous system of the subject is the brain tissue of the subject.

### Embodiment 45, Brain Tissue Only

The method of embodiment 44, wherein the brain tissue of the subject is selected from the group consisting of Substantia Nigra, Cerebral Ventricle, and Striatum.

### Embodiment 46, Contact Induction Method Only

The method of any one of embodiments 41 to 45, wherein inducing the contact is by injecting the composition into the subject.

### Embodiment 47, Example Contact Induction Method

The method of embodiment 46, wherein the injection is selected from an intracerebral injection, and an intracerebroventricular injection (ICV).

### Treatments for Neurodeqenerative Diseases

### Embodiment 48, Method of Treating a Neurodegenerative Disease

Treatments for neurodegenerative diseases, including: Administering the composition of any one of embodiments 35 to 37 to a subject.

### Embodiment 49, Indication-Limited

The treatment method of embodiment 48, wherein the neurodegenerative disease is selected from the group consisting of Alzheimer's disease, Parkinson's disease, Lewy bodies disease, Pick's disease, Traumatic encephalopathy, Amyotrophic Lateral Sclerosis; ALS), Tauopathy, and Frontotemporal dementia.

### Embodiment 50, Site-Specific Administration

The method of treatment of any one of embodiments 50 to 49, wherein the administration is to the central nervous system of the subject.

### Embodiment 51, Brain Dosing Only

The method of treatment of embodiment 50, wherein the central nervous system is the brain tissue of the subject.

### Embodiment 52, Brain Tissue Only

The method of treatment of embodiment 51, wherein the brain tissue of the subject is selected from the Substantia Nigra, the Cerebral Ventricle, and the Striatum.

### Embodiment 53, Brain Administration Method Only

The method of treatment of any one of embodiments 51 to 52, wherein the administration is by a method selected from the intracerebral injection, and intracerebroventricular injection (ICV).

### Uses of a Composition Comprising a Prion-Fc Region Fusion Protein or a Vector Capable of Expressing the Same

### Embodiment 54, Therapeutic Use of the Composition

Use of the composition of any one of embodiments 35 to 37 in the manufacture of a treatment for neurodegenerative disease.

### Embodiment 55, Indication-Limited

In embodiment 54, the neurodegenerative disease is selected from the group consisting of Alzheimer's disease, Parkinson's disease, Lewy bodies disease, Pick's disease, Traumatic encephalopathy, Amyotrophic Lateral Sclerosis; ALS), Tauopathy, and Frontotemporal dementia.

### Embodiment 56, Use of the Composition to Remove Neurotoxic Protein Aggregates

Use of the composition of any one of embodiments 35 to 37 to remove a neurotoxic protein aggregate in a subject.

### Embodiment 57, Limiting the Class of Neurotoxic Protein Aggregates

The use of embodiment 56, wherein the neurotoxic protein aggregates are one or more selected from the group consisting of alpha-synuclein aggregates, amyloid beta aggregates, tau aggregates, TDP-43 aggregates, and prion aggregates.

### MODE FOR INVENTION

Hereinafter, the invention provided by the present specification will be described in more detail through Experimental Examples and Examples. These Examples are only for the purpose of illustrating the contents disclosed by the present specification, and it will be apparent to those skilled in the art that the scope of the contents disclosed by the present specification is not to be construed as being limited by these Examples.

### Experimental Example 1 Material and Methods

### Experimental Example 1.1 AAV Vector Preparation

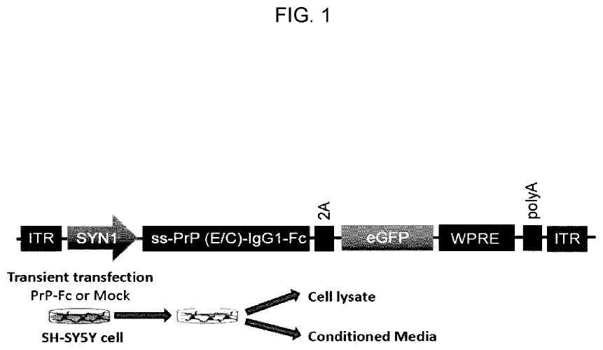

The IL-2ss-hPrP-Fc expression vector was constructed from an AAV-backbone plasmid. The expression vector is shown schematically in FIG. 1, and the respective sequences contained in the vector are shown in Table 1.

TABLE 1

| | | | SEQ ID |
|---|---|---|---|
| Label | Description | Sequence (5′ to 3′) | NO |
| hSYN1 | Promoter | ctgcagagggccctgcgtatgagtgcaagtgggttttaggaccaggat | 60 |
| | | gaggcgggggggggtgcctacctgacgaccgaccccgacccactg | |
| | | gacaagcacccaacccccattccccaaattgcgcatcccctatcaga | |
| | | gagggggaggggaaacaggatgcggcgaggcgcgtgcgcactgc | |
| | | cagcttcagcaccgcggacagtgccttcgcccccgcctggcggcgcg | |
| | | cgccaccgccgcctcagcactgaaggcgcgctgacgtcactcgccg | |
| | | gtcccccgcaaactccccttcccggccaccttggtcgcgtccgcgccg | |
| | | ccgccggcccagccggaccgcaccacgcgaggcgcgagataggg | |
| | | gggcacgggcgcgaccatctgcgctgcggcgccggcgactcagcgc | |
| | | tgcctcagtctgcggtgggcagcggaggagtcgtgtcgtgcctgagag | |
| | | cgcag | |
| SS | Secretion | atgtacaggatgcaactcctgtcttgcattgcactaagtcttgcacttgtca | 61 |
| | signal | cgaattcg | |
| | peptide | | |
| PrP(E/C) | human | atggcgaaccttggctgctggatgctggttctctttgtggccacatggagt | 54 |
| (WT) | prion | gacctgggcctctgcaagaagcgcccgaagcctggaggatggaaca | |
| | protein | ctgggggcagccgatacccggggcagggcagccctggaggcaacc | |
| | | gctacccacctcagggcggtggtggctgggggcagcctcatggtggtg | |
| | | gctgggggcagcctcatggtggtggctgggggcagccccatggtggt | |
| | | ggctggggacagcctcatggtggtggctggggtcaaggaggtggcac | |
| | | ccacagtcagtggaacaagccgagtaagccaaaaaccaacatgaa | |
| | | gcacatggctggtgctgcagcagctggggcagtggtggggggccttg | |
| | | gcggctacatgctgggaagtgccatgagcaggcccatcatacatttcg | |
| | | gcagtgactatgaggaccgttactatcgtgaaaacatgcaccgttaccc | |
| | | caaccaagtgtactacaggcccatggatgagtacagcaaccagaac | |
| | | aactttgtgcacgactgcgtcaatatcacaatcaagcagcacacggtc | |
| | | accacaaccaccaagggggagaacttcaccgagaccgacgttaag | |
| | | atgatggagcgcgtggttgagcagatgtgtatcacccagtacgagagg | |
| | | gaatctcaggcctattaccagagaggatcgagcatggtcctcttctcctc | |
| | | tccacctgtgatcctcctgatctctttcctcatcttcctgatagtggga | |
| IgG1-Fc | human | gacaaaactcacacatgcccaccgtgcccagcacctgaactcctggg | 62 |
| | IgG1 | gggaccgtcagtcttcctcttccccccaaaacccaaggacaccctcat | |
| | Fc region | gatctcccggacccctgaggtcacatgcgtggtggtggacgtgagcca | |
| | | cgaagaccctgaggtcaagttcaactggtacgtggacggcgtggagg | |
| | | tgcataatgccaagacaaagccgcgggaggagcagtacaacagca | |
| | | cgtaccgtgtggtcagcgtcctcaccgtcctgcaccaggactggctgaa | |
| | | tggcaaggagtacaagtgcaaggtctccaacaaagccctcccagcc | |
| | | cccatcgagaaaaccatctccaaagccaaagggcagccccgagaa | |
| | | ccacaggtgtacaccctgcccccatcccgggaggagatgaccaaga | |
| | | accaggtcagcctgacctgcctggtcaaaggcttctatcccagcgacat | |
| | | cgccgtggagtgggagagcaatgggcagccggagaacaactacaa | |
| | | gaccacgcctcccgtgctggactccgacggctccttcttcctctacagca | |
| | | agctcaccgtggacaagagcaggtggcagcaggggaacgtcttctca | |
| | | tgctccgtgatgcacgaggctctgcacaaccactacacgcagaagag | |
| | | cctctccctgtctccgggtaaa | |
| eGFP | Green | atggtgagcaagggcgaggagctgttcaccggggtggtgcccatcct | 63 |
| | fluorescent | ggtcgagctggacggcgacgtaaacggccacaagttcagcgtgtccg | |
| | protein | gcgagggcgagggcgatgccacctacggcaagctgaccctgaagtt | |
| | | catctgcaccaccggcaagctgcccgtgccctggcccaccctcgtgac | |
| | | caccctgacctacggcgtgcagtgcttcagccgctaccccgaccacat | |
| | | gaagcagcacgacttcttcaagtccgccatgcccgaaggctacgtcca | |
| | | ggagcgcaccatcttcttcaaggacgacggcaactacaagacccgc | |
| | | gccgaggtgaagttcgagggcgacaccctggtgaaccgcatcgagct | |
| | | gaagggcatcgacttcaaggaggacggcaacatcctggggcacaa | |
| | | gctggagtacaactacaacagccacaacgtctatatcatggccgaca | |
| | | agcagaagaacggcatcaaggtgaacttcaagatccgccacaacat | |
| | | cgaggacggcagcgtgcagctcgccgaccactaccagcagaacac | |
| | | ccccatcggcgacggccccgtgctgctgcccgacaaccactacctga | |
| | | gcacccagtccgccctgagcaaagaccccaacgagaagcgcgatc | |
| | | acatggtcctgctggagttcgtgaccgccgccgggatcactctcggcat | |
| | | ggacgagctgtacaagtccggataa | |

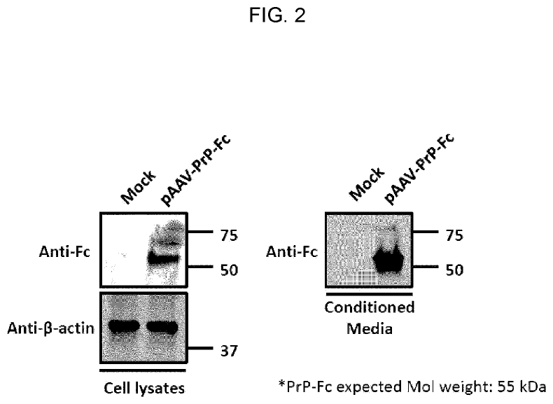

At this time, the constitutive integrity of the plasmid was confirmed by sequencing. The plasmid was prepared by amplifying an E. coli-based miniprep. The expression and secretion efficiency for human prion protein in the conditioned culture medium was verified by Western blot analysis according to Experimental example 1.9, which is shown in FIG. 2.

### Experimental Example 1.2 SH-SY5Y Cell Preparation

Human neuroblastoma SH-SY5Y cells were grown in DMEM containing 10% (v/v) fetal bovine serum (FBS) and antibiotics at 37° C. in a humidified 5% CO₂ atmosphere.

### Experimental Example 1.3 Preparation of Culture Medium Containing Prion-Fc Fusion Protein

To obtain an α-synuclein-regulated medium secreted by neurons, SH-SY5Y cells from Experimental example 1.2 were transfected with pAAV-IL-2ss-hPrP-Fc prepared in Experimental example 1.1. Specifically, Lipofectamine 2000 transfection reagent (Thermo-Fisher) was used for transient transfection according to the manufacturer's instructions. Two days after transfection, the culture medium was replaced with serum-free Dulbecco's modified Eagle's medium (DMEM) and incubated for an additional 18 hours. To remove cellular debris, the medium was centrifuged at 10,000 g for 10 minutes. After centrifugation, the recovered supernatant was concentrated using a 10K cut-off centrifugation filter.

### Experimental Example 1.4 Preparation of Preformed Fibrils (PFF)

Alpha-synuclein fibrils (50 μM), prepared by incubating monomeric alpha-synuclein for 7 days, were incubated_in vitro_in sodium acetate buffer (100 mM, pH 7.5) with or without PCIII (100 μM) in a shaking incubator (300 rpm; Vision Scientific) at 37° C. Samples were taken on days 1, 3, and 7 of incubation, respectively, and thioflavin T fluorescence readings were performed.

### Experimental Example 1.5 Make a Lewy Body Model Mouse

AAV-alpha-synuclein expressing alpha-synuclein was injected into mice by intraperitoneal injection. The AAV-alpha-synuclein was performed in the SN (anteroposterior, 3.2 mm from bregma; mediolateral, 1.3 mm; dorsoventral, 4.3 mm) and VTA (anteroposterior, 3.2 mm from bregma; mediolateral, 0.5 mm; dorsoventral 4.3 mm) locations. Four days after viral injection, alpha-synuclein PFF (5 ug/2 ul) was stereotaxically injected into the above SN and VTA coordinates. After the final injection, the infusion cannula was kept in the striatum or VTA for an additional 5 minutes to allow for complete absorption of the chemical or virus, and then slowly removed from the rat brain.

### Experimental Example 1.6 CCK-8 Cell Viability Assay

Target cells were plated in 96-well white flat-bottom plates (Microtiter; Thermo Scientific, USA) at 80% confluence in 100 μl of DMEM containing penicillin/streptomycin (P/S) and 10% FBS. Cell viability was assessed using the CCK-8 assay.

### Experimental Example 1.7 Fluorescence Imaging of Target Cells

The target cells were plated at 10,000 cells/cm2 on poly-L-lysine-coated coverslips. Thereafter, the cells were fixed with 4% paraformaldehyde in PBS and blocked in a solution containing 5% normal goat serum, 2% BSA (Sigma), and 0.1% Triton X-100 (Sigma) for 1 hour at room temperature. Subsequently, the cell samples were incubated with primary antibodies corresponding to the protein of interest overnight at 4° C.

Subsequently, cells grown on the coverslips were washed with PBS containing 0.1% Triton X-100 and incubated with fluorescently-conjugated secondary antibodies (1:500; Invitrogen) for 1 hour at room temperature. The coverslips were mounted with 4′, 6-diamidino-2-phenylindole (DAPI). A confocal microscope was used for four-channel fluorescence image acquisition.

### Experimental Example 1.8 Fluorescence Imaging of Mouse Brain Tissue

Mice were anesthetized, perfused with PBS, post-fixed with 4% paraformaldehyde in PBS for 24 hours, and embedded in 30% sucrose in PBS. Coronal sections of 35 μm were then cut, and every fourth section was used for analysis. Brain sectioned cells were blocked in blocking buffer [5% (v/v) goat serum in PBS and 0.1% (v/v) Triton X-100 in PBS] with primary antibodies (anti-tyrosine hydroxylase, novus biologicals, NB300-109 or anti-pS129-a-synuclein, biolegend, 825701) overnight at 4° C. and incubated for 1 hour at 25° C. with appropriate fluorescence-conjugated secondary antibodies (Alexa Fluor 488/594-anti rabbit or mouse IgG).

Immunofluorescence images of the above brain sections were obtained with a Carl Zeiss confocal microscope. TH or pS129-a-synuclein-positive signals in the above immunofluorescence images were performed according to the software protocol of ImageJ. The immunofluorescence images of TH (green) or pS129-a-synuclein-positive signal (red) were converted to grayscale, the threshold for immunostaining was set according to the particle size, and the area of TH, or pS129-a-synuclein-positive signal, was defined. Based on the above information, the fluorescence intensity values were finally measured and organized.

### Experimental Example 1.9 Western Blot Analysis

For Triton X-100-soluble and -insoluble compartmentalization, target cells were harvested and treated with non-ionic detergent-soluble and detergent-insoluble fractions in lysis buffer containing a mixture of PBS, 1% Triton X-100, Phosphatase Inhibitor Cocktail II, III, and a complete protease inhibitor. The lysate was centrifuged at 100,000 g, 4° C. for 20 minutes. After centrifugation, the resulting pellet and supernatant (S1, soluble) fraction were collected. The pellet was washed once in lysis buffer containing nonionic detergent (1% Triton X-100) and dissolved in lysis buffer containing 1% SDS and 0.5% sodium deoxycholate. The homogenate was centrifuged, and the resulting supernatant (non-ionic detergent-insoluble) was collected.

For total lysates, cells were harvested, washed twice with PBS, and lysed in Pierce RIPA buffer (150 mM NaCl, 50 mM Tris, pH 8.0, 1% NP40, 1% SDS, and 0.5% sodium deoxycholate, and a protease inhibitor mixture; Thermo Scientific) for 30 min on ice. The cell lysates were then centrifuged at 22,250 g, 4° C. for 20 min. Protein concentration was measured using a BCA protein assay kit (Thermo Scientific). Equal amounts of protein (10-20 μg) were resolved on 8% to 16% SDS-PAGE and transferred to nitrocellulose (NC) membranes. After washing with TBST (Tris-buffer solution-Tween20; 10 mM Tris-HCl [pH 7.6], 150 mM NaCl, and 0.05% Tween-20), the NC membrane was blocked with 5% skim milk for 1 h and incubated in the appropriate dilution of primary antibody recommended by the supplier. The NC membrane was then washed, and the primary antibody was detected with an HRP-conjugated secondary antibody. Immunoblot signals were visualized by Chemiluminescence (Pierce, USA). Densitometric analyses of immunoreactive bands were performed with NIH ImageJ software. The ratio between treated and control samples was calculated for each experiment and expressed as a relative value to the control.

### Experimental Example 2 Evaluation of In Vitro Efficacy of Prion-Fc Fusion Protein Against SH-SY5Y Cells

### Experimental Example 2.1 Preparation of Embodiments for In Vitro Efficacy Evaluation of Prion-Fc Fusion Proteins Against SH-SY5Y Cells

For SH-SY5Y cells cultured according to Experimental example 1.2, each embodiment was prepared by culturing them under different culture conditions. The conditions for each embodiment are shown in Table 2.

TABLE 2

| | Alpha-synuclein | PrP-Fc |
|---|---|---|
| Label | PFF (μg/ml) | (ng/ml) |
| Comparative example 2.1 | 0 | 0 |
| Comparative example 2.2 | 0 | 100 |
| Comparative example 2.3 | 5 | 0 |
| Example 2.1 | 5 | 100 |

### Experimental Example 2.2 Viability Assessment for SH-SY5Y Cells (CCK-8 Assay)

SH-SY5Y cells of each embodiment according to Experimental example 2.1 were evaluated for cell viability by CCK-8 assay according to Experimental example 1.6.

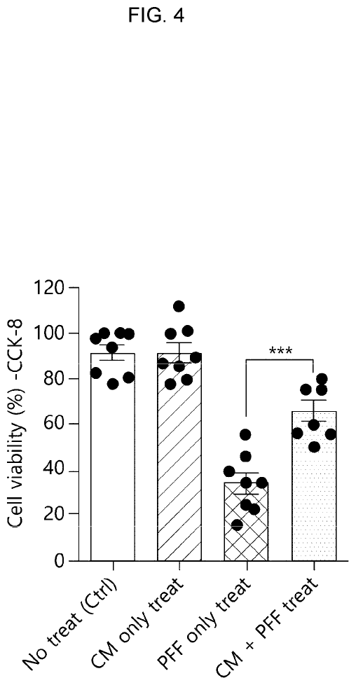

The results of the viability assessment are shown in FIG. 4. The results of the viability assessment showed that SH-SY5Y cells cultured in the presence of prion-Fc fusion protein (Example 2.1) had a significantly higher survival rate compared to SH-SY5Y cells cultured in the presence of alpha-synuclein PFF alone (Comparative Example 2.3).

### Experimental Example 2.3 Observe Immunofluorescence Images for SH-SY5Y Cells

Immunofluorescence images were obtained according to Experimental example 1.7 for cells according to each of the above embodiments. In Experimental example 1.8, the subject cells were SH-SY5Y cells according to each of the above embodiments, and the primary antibodies were pS129—α-Synuclein (abcam, ab51253); tyrosine hydroxylase (Novus Biologicals, NB300-109).

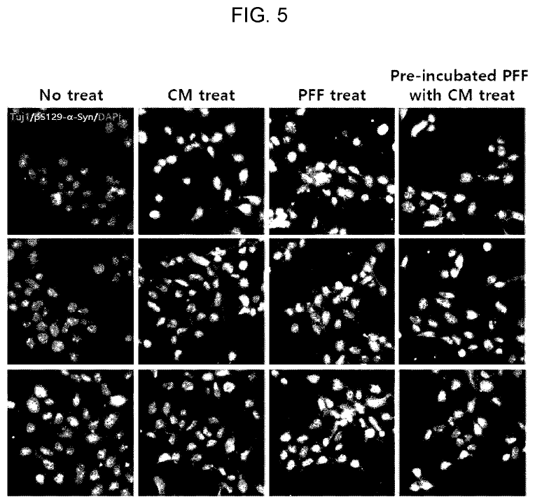

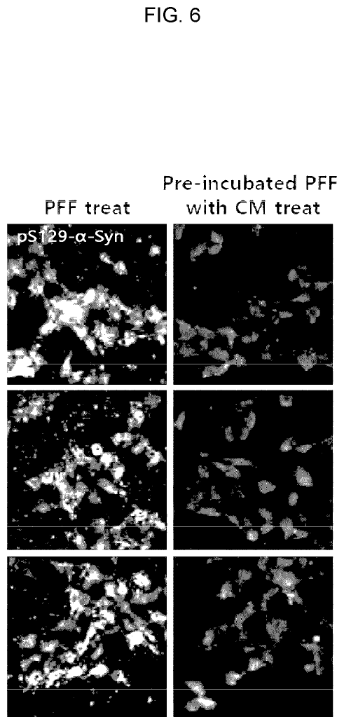

The obtained immunofluorescence images are shown in FIGS. 5 and 6.

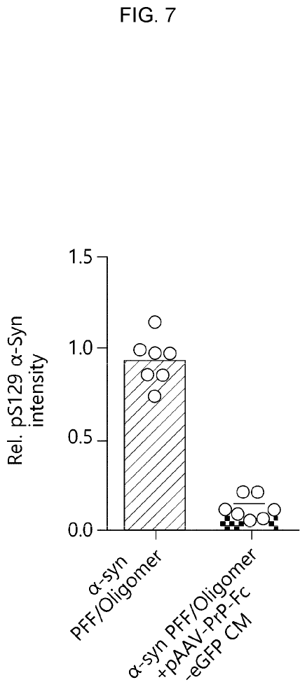

In the above immunofluorescence images, the areas of fluorescence represent the prion-Fc region fusion protein, alpha-synuclein PFF, and oligomers around the SH-SY5Y cells. Comparison of the relative fluorescence intensity for pS219 alpha-synuclein in the above immunofluorescence images shows that the alpha-synuclein-labeled fluorescence around pS219 of SH-SY5Y cells (Example 2.1) cultured in the presence of prion-Fc fusion protein is significantly lower (FIG. 7).

### Experimental Example 3 Evaluation of Microglia Activity of Prion-Fc Region Fusion Proteins

### Experimental Example 3.1 Preparation of an Example to Evaluate the Effect of Prion-Fc Fusion Protein on Microglia Activity

To evaluate the effect of the prion-Fc fusion protein on microglial activity, embodiments were prepared with different culture conditions. The conditions according to the embodiments are shown in Table 3.

TABLE 3

| | Alpha-synuclein | PrP-Fc | Incubation |
|---|---|---|---|
| Label | PFF (μg/ml) | (ng/ml) | Time (hrs) |
| Comparative example 3.1 | 5 | 0 | 0.5 |
| Comparative example 3.2 | 5 | 0 | 1 |
| Comparative example 3.3 | 5 | 0 | 3 |
| Comparative example 3.4 | 5 | 0 | 5 |
| Comparative example 3.5 | 5 | 0 | 8 |
| Comparative example 3.6 | 5 | 0 | 10 |
| Comparative example 3.7 | 5 | 0 | 12 |
| Example 3.1 | 5 | 100 | 0.5 |
| Example 3.2 | 5 | 100 | 1 |
| Example 3.3 | 5 | 100 | 3 |
| Example 3.4 | 5 | 100 | 5 |
| Example 3.5 | 5 | 100 | 8 |
| Example 3.6 | 5 | 100 | 10 |
| Example 3.7 | 5 | 100 | 12 |

### Experimental Example 3.2 Observe Immunofluorescence Images for SIM-A9 Cells

For each embodiment according to Experimental example 3.1, immunofluorescence images were obtained according to Experimental example 1.7. In Experimental example 1.8, the subject cells were SIM-A9 cells according to each embodiment and the primary antibodies were Iba-I (abcam, ab15690); α-Synuclein (BD bioscience, #610787).

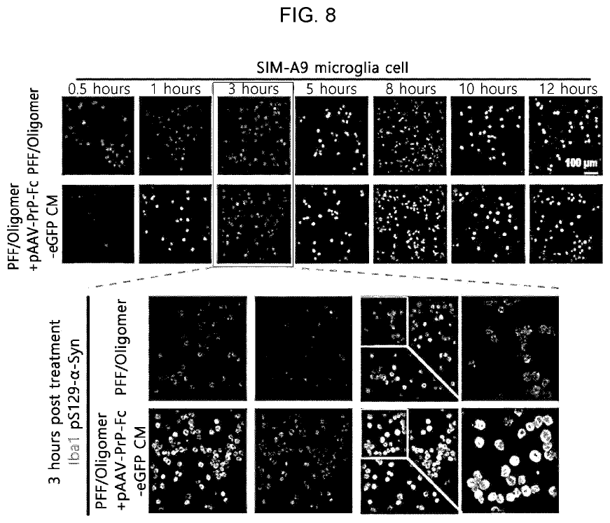

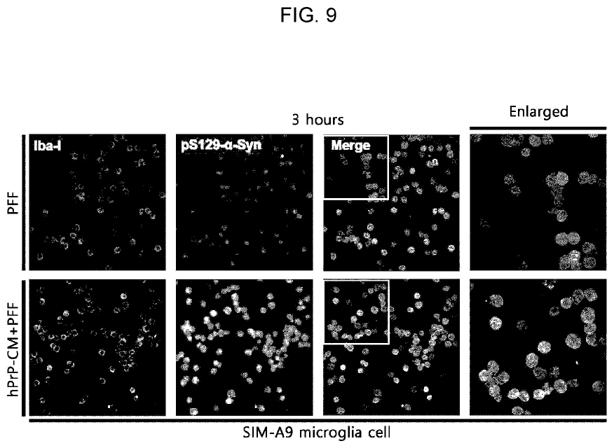

The obtained immunofluorescence images are shown in FIGS. 8 and 9.

In the immunofluorescence image above, the fluorescent area represents alpha-synuclein incorporated into SIM-A9 microglia via macrophagy.

### Experimental Example 3.3 Activity Evaluation on SIM-A9 Cells

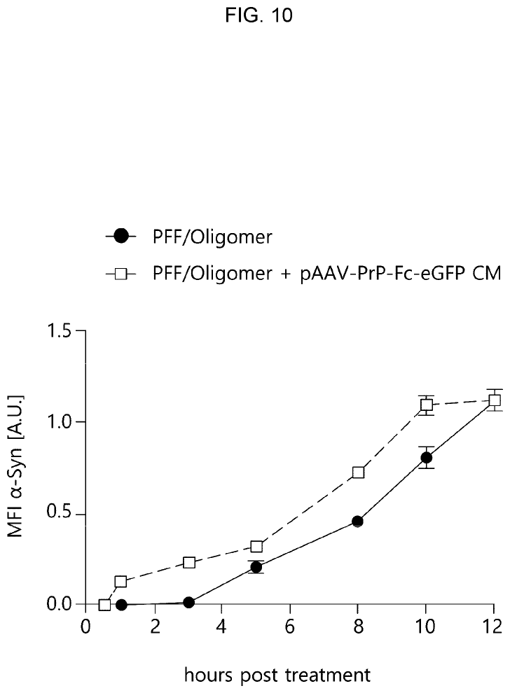

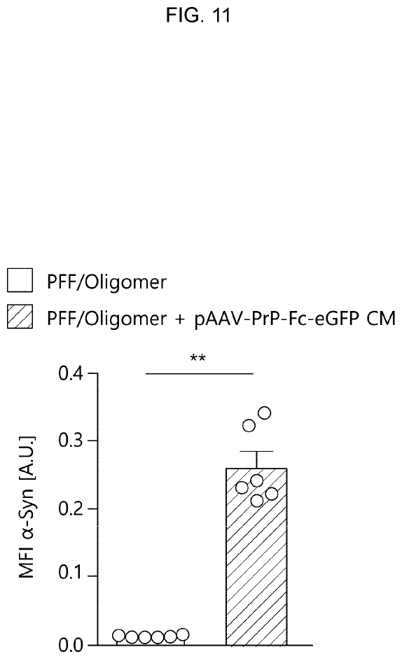

The immunofluorescence images according to Experimental example 3.2 were analyzed to determine the effect of the prion-Fc region fusion protein on the activity of SIM-A9 cells. The average fluorescence intensity for alpha-synuclein of Examples EX01 to EX02 was measured and is shown in FIG. 10 for SIM-A9 cells treated with alpha-synuclein alone and SIM-A9 cells treated with both alpha-synuclein and prion-Fc fusion protein. The average fluorescence intensity for alpha-synuclein of Example EX03 and EX04 was measured and is shown in FIG. 11 for SIM-A9 cells added only alpha-synuclein and SIM-A9 cells added both alpha-synuclein and prion-Fc fusion protein.

The results showed that in the presence of both alpha-synuclein PFF and prion-Fc fusion proteins, SIM-A9 cells rapidly developed macrophages against alpha-synuclein PFF.

### Experimental Example 4 Evaluation of In Vivo Efficacy of Prion-Fc Fusion Proteins In Vivo Lewy Body Mouse Model

### Experimental Example 4.1 Injecting an AAV Vector Expressing a Prion-Fc Fusion Protein into a Model Mouse

To determine the effect of the prion-Fc fusion protein, stereotaxic injection of AAV-PrP-Fc virus capable of expressing the prion-Fc fusion protein was performed in a Lewy body model mouse prepared according to Experimental example 1.5. Specifically, an injection cannula (26.5 gauge) was inserted into the striatum (intrastriatal: anteroposterior, 0.5 mm from bregma; mediolateral, 2.0 mm; dorsoventral, 3.0 mm), within SN coordinates (intranigral injection: anteroposterior, 3.2 mm from bregma; mediolateral, 1.3 mm; dorsoventral, 4.3 mm), or in the ventricular zone (For intracerebroventricular: anteroposterior, −1.0 mm, mediolateral, 0.6 mm, dorsoventral, 2.0 mm from bregma) to stereotaxically inject AAV-PrP-Fc virus.

The embodiments used in Experimental example 4 are shown in the following Table 4.

TABLE 4

| | | AAF-PrP-Fc |
|---|---|---|
| | | injection |
| Label | injection Location | volume |
| Comparative | — | — |
| example 4.1 (WT) | | |
| Comparative | — | — |
| example 4.2 (LB) | | |
| Example 4.1 (STR) | intrastriatal, | 1E10 vg |
| | 0.5 mm from begma, | (5E11 vg/kg) |
| | mediolateral 2.0 mm, | |
| | dorsoventral 3.0 mm. | |
| Example 4.2 (ICV) | intranigral injection, | 1E10 vg |
| | 3.2 mm from begma, | (5E11 vg/kg) |
| | mediolateral 1.3 mm, | |
| | dorsoventral 4.3 mm. | |
| Example 4.3 (SNpc) | intracerebroventricular, | 1E10 vg |
| | 2.0 mm from begma, | (5E11 vg/kg) |
| | anteroposterior −1.0 mm, | |
| | mediolateral 0.6 mm. | |

### Experimental Example 4.2 Pole Test

For the pole test, each mouse according to the embodiment in Experimental example 4.1 was acclimatized to the behavioral procedure cage for at least 30 minutes. The pole was a metal rod with a diameter of 10 mm and a length of 58 cm, with bandage gauze wrapped around the pole. The pole was placed in the behavioral procedure cage, and the mouse was placed on the top part of the pole with its head facing down. The total time it took for the mouse to reach the bottom of the pole was then recorded. This was evaluated three times and the average time was recorded.

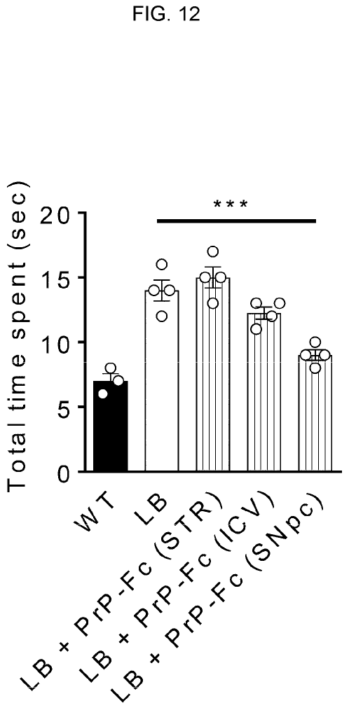

The results of the above Paul test are shown in FIG. 12. The Paul test results showed that when the prion-Fc fusion protein expression vector was injected into the substantia nigra pars compacta (SNpc) region of the mouse (Example 4.3), the time taken was close to that of a normal mouse (Comparative Example 4.1).

### Experimental Example 4.3 Hindlimb Extensor Reflex Test

For hind limb extensor testing, a mouse according to each of the above embodiments was suspended by its tail and its hind limb extensor reflexes were recorded on a scale of 0 to 2 (0: complete paralysis; 0.5: no hind limb extensor reflexes; 1.0: extensor reflexes on one hind limb only; 1.5: unbalanced extensor reflexes of the hind limbs; 2.0: normal extensor reflexes of both hind limbs).

### Experimental Example 4.4 Western Blot Analysis of Mouse Brain Tissue

Brains from each of the above embodiment mice were analyzed by Western blot according to Experimental example 1.9.

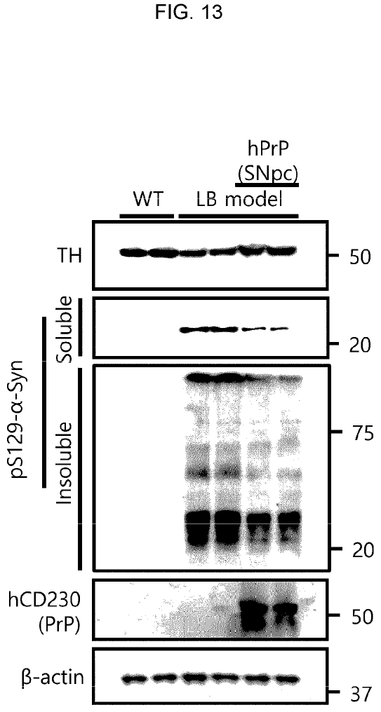

The results of the western blot analysis are shown in FIG. 13.

### Experimental Example 4.5 Observe Immunofluorescence Images of Mouse Brain Tissue

The brains of each of the above embodiment mice were fluorescence imaged according to Experimental example 1.8.

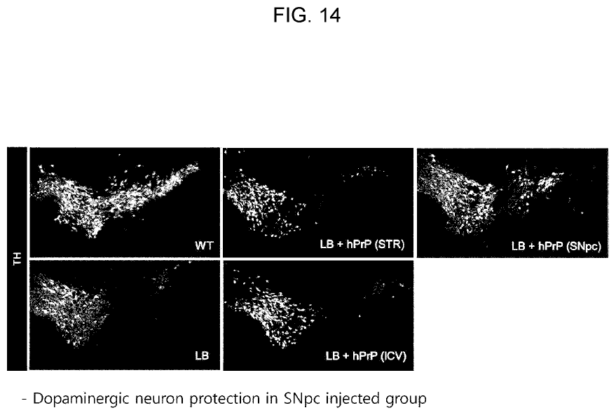

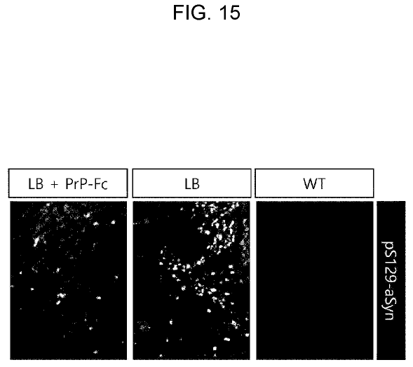

The obtained immunofluorescence images are shown in FIGS. 14 and 15. From the observations, it can be seen that when the prion-Fc region fusion protein expressing AAV vector was injected into the substantia nigra pars compacta (SNpc) region of the mouse (Example 4.3), the dopaminergic neurons of the mouse were well protected compared to the results of the Lewy body model mouse (Comparative Example 4.2).

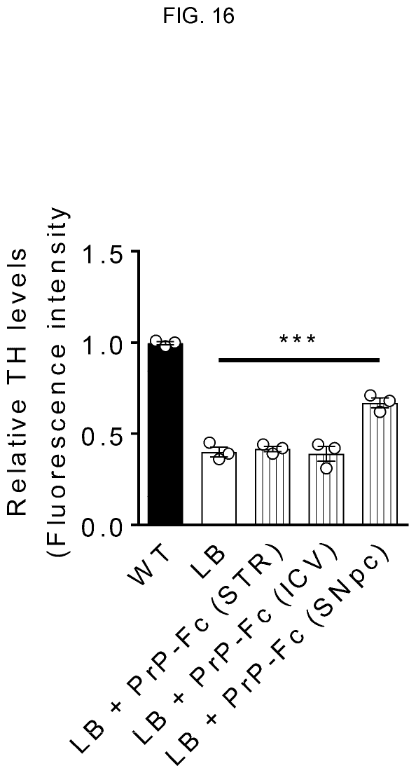

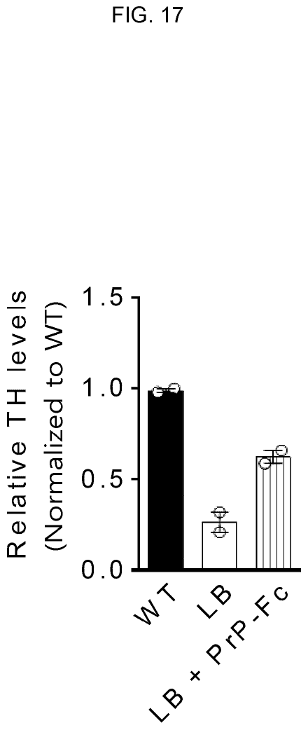

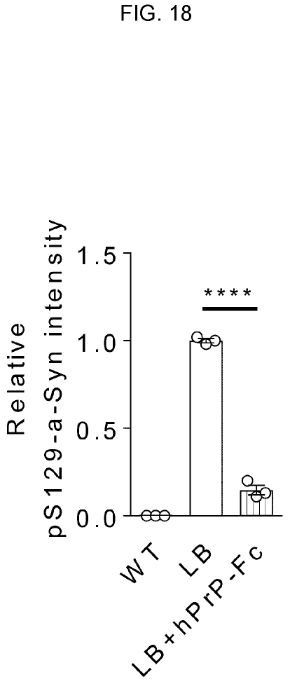

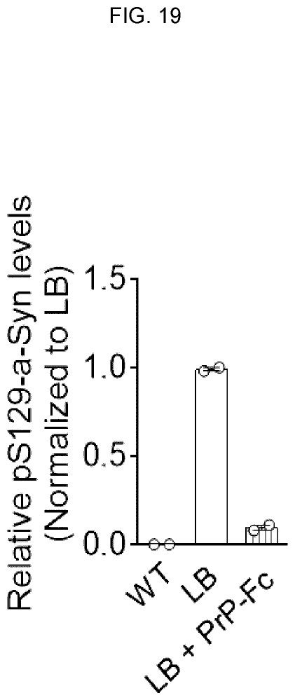

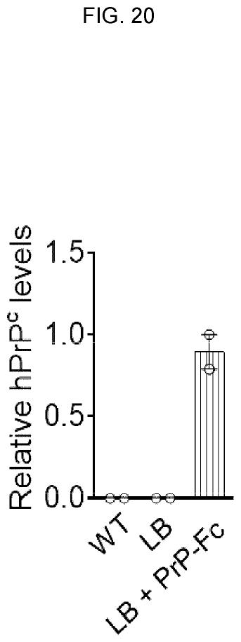

The results of measuring the fluorescence intensity according to each embodiment are shown in FIGS. 16 to 20. The measurements showed that the relative TH fluorescence intensity was significantly higher when injected into the substantia nigra pars compacta (SNpc) region of the mouse (Example 4.3, SNpc) compared to the results in the Louisbody model mouse (Comparative Example 4.2, LB), indicating that the dopaminergic neurons in the mouse were viable (FIGS. 16 and 17). On the other hand, for pS219 alpha-synuclein-labeled fluorescence, the fluorescence signal was significantly reduced in mice injected with the AAV vector expressing the prion-Fc region compared to the Lewy body model mice (Comparative Example 4.2, LB), indicating that the prion-Fc region has an alpha-synuclein aggregate removal effect (FIG. 18 and FIG. 19).

### Experimental Example 5 Evaluation of In Vitro Efficacy for Human Prion Protein Variants and Fragments 1

### Experimental Example 5.1 Preparation of Prion-Fc Region Fusion Proteins Containing Human Prion Protein Variants and Fragments

For in vitro efficacy evaluation of prion-Fc region fusion proteins comprising human prion protein fragments, variants, or fragments of variants, vectors expressing prion-Fc fusion proteins were first prepared according to Experimental example 1.1, except that the human prion proteins listed as PrP(E/C) in Table 1 were replaced as shown in the following Table 5.

TABLE 5

| | PrP peptide | | SEQ ID |
|---|---|---|---|
| Label | sequence | PrP encoding DNA sequence (5′ to 3′) | NO |
| F1 | SEQ ID NO: 6 | cctcagggcggtggtggctgggggcagcctcatggtggtgg | 55 |
| | | ctgggggcagcctcatggtggtggctgggggcagccccat | |
| | | ggtggtggctggggacagcctcatggtggtggctggggtca | |
| | | aggaggtggcacccacagtcagtggaacaagccgagtaa | |
| | | gccaaaaaccaacatgaagcacatggctggtgctgcagc | |
| | | agctggggcagtggtggggggccttggcgtttacatgctggg | |
| | | aagtgccatg | |
| F2 | SEQ ID NO: 2 | cctcagggcggtggtggctgggggcagcctcatggtggtgg | 56 |
| | | ctgggggcagcctcatggtggtggctgggggcagccccat | |
| | | ggtggtggctggggacagcctcatggtggtggctggggtca | |
| | | aggaggtggcacccacagtcagtggaacaagccgagtaa | |
| | | gccaaaaaccaacatgaagcac | |
| F3 | SEQ ID NO: 4 | aagaagcgcccgaagcctggaggatggaacactggggg | 57 |
| | | cagccgatacccggggcagggcagccctggaggcaacc | |
| | | gctacccacctcagggcggtggtggctgggggcagcctcat | |
| | | ggtggtggctgggggcagcctcatggtggtggctgggggca | |
| | | gccccatggtggtggctggggacagcctcatggtggtggct | |
| | | ggggtcaaggaggtggcacccacagtcagtggaacaagc | |
| | | cgagtaagccaaaaaccaacatgaagcac | |
| F4 | SEQ ID NO: 9 | aagaagcgcccgaagcctggaggatggaacactggggg | 58 |
| | | cagccgatacccggggcagggcagccctggaggcaacc | |
| | | gctacccacctcagggcggtggtggctgggggcagcctcat | |
| | | ggtggtggctgggggcagcctcatggtggtggctgggggca | |
| | | gccccatggtggtggctggggacagcctcatggtggtggct | |
| | | ggggtcaaggaggtggcacccacagtcagtggaacaagc | |
| | | cgagtaagccaaaaaccaacatgaagcacatggctggtg | |
| | | ctgcagcagctggggcagtggtggggggccttggcgtttac | |
| | | atgctgggaagtgccatg | |
| F5 | SEQ ID NO: 10 | acccacagtcagtggaacaagccgagtaagccaaaaacc | 59 |
| | | aacatgaagcacatggctggtgctgcagcagctggggcag | |
| | | tggtggggggccttggcgtttacatgctgggaagtgccatg | |

Prepare the alpha-synuclein conditioned medium according to Experimental example 1.3, but transfect the SH-SY5Y cells of Experimental example 1.2 with the AAV vectors according to Table 5 to prepare the culture medium containing the respective prion-Fc fusion proteins.

For the in vitro efficacy evaluation of prion-Fc region fusion proteins comprising human prion protein fragments, variants, or fragments of variants, the following embodiments were prepared with different culture conditions for each embodiment, as shown in Table 6.

TABLE 6

| | Alpha-synuclein | PrP-Fc | PrP-Fc |
|---|---|---|---|
| Label | PFF (μg/ml) | type | (ng/ml) |
| Comparative Example 5.1 | — | — | 0 |
| Comparative Example 5.2 | 5 | — | 0 |
| Comparative Example 5.3 | 5 | WT | 100 |
| Example 5.1 | 5 | F1 | 100 |
| Example 5.2 | 5 | F2 | 100 |
| Example 5.3 | 5 | F3 | 100 |
| Example 5.4 | 5 | F4 | 100 |
| Example 5.5 | 5 | F5 | 100 |

### Experimental Example 5.2 Identify Human Prion Protein Variants and Cleavage Expression

For each embodiment according to Experimental example 5.1, a western blot analysis was performed according to Experimental example 1.10. In Experimental example 1.10, the subject cells were SH-SY5Y cells according to each embodiment.

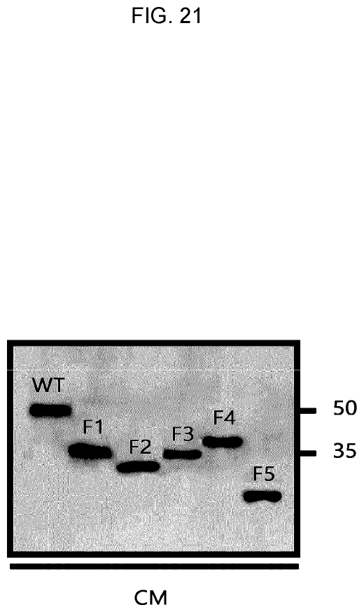

The results of the western blot analysis are shown in FIG. 21.

### Experimental Example 5.3 Evaluate In Vitro Efficacy on SH-SY5Y Cells

For each embodiment according to Experimental example 5.1, cell viability was assessed by CCK-8 assay according to Experimental example 1.7.

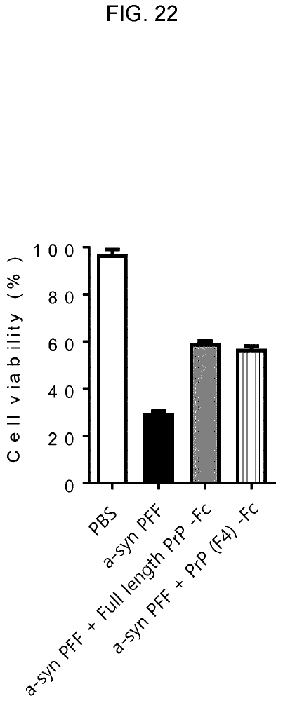

The results of the survival rate evaluation are shown in FIG. 22. As a result of the viability evaluation, the viability of SH-SY5Y cells in the presence of the prion-Fc region fusion protein of Example 5.4 was similar to the viability of SH-SY5Y cells in the presence of the prion-Fc region fusion protein of Comparative Example 5.3. Therefore, it can be seen that the prion-Fc region fusion protein comprising a fragment of human prion protein and its variants has the same effect as the prion-Fc region fusion protein comprising wild-type human prion protein.

### Experimental Example 6 Evaluating In Vitro Efficacy on Primary Hippocampal

### Experimental Example 6.1 Prepare Primary Hippocampal Neuron Cells

Neural progenitor cells (NPCs) were isolated from the hippocampus dissected from mouse embryos (P1 to P2). The isolated neural progenitor cells were plated on glass coated with poly-L-ornithine and allowed to proliferate in serum-supplemented neural cell culture medium (37° C.). The neural progenitor cells were proliferated for 3 days and replaced with fresh culture medium every day. The neural progenitor cells were then cultured in Serum-free Neuronal Culture-Medium (37° C.) with Neuronal Growth Supplement for 5 to 7 days to differentiate into neurons.

### Experimental Example 6.2 Preparation of Prion-Fc Region Fusion Proteins Containing Human Prion Protein Variants and Fragments

For the evaluation of the in vitro efficacy against alpha-synuclein PFF and tau protein aggregates of prion-Fc region fusion proteins comprising human prion protein fragments, variants, or fragments of variants, the embodiments shown in Table 7 below were prepared concerning Experimental example 5.1.

TABLE 7

| | | Neurotoxic | | PrP-Fc |
|---|---|---|---|---|
| | Neurotoxic | protein added | PrP-Fc | added |
| Label | protein type | (μg/ml) | type | (ng/ml) |
| Comparative | — | — | — | — |
| example 6.1 | | | | |
| Comparative | — | — | — | — |
| example 6.2 | | | | |
| Comparative | alpha-synuclein | 5 | — | — |
| example 6.3 | PFF | | | |
| Comparative | tau PFF | 5 | — | — |
| example 6.4 | | | | |
| Example 6.1 | alpha-synuclein | 5 | WT | 100 |
| | PFF | | | |
| Example 6.2 | alpha-synuclein | 5 | F4 | 100 |
| | PFF | | | |
| Example 6.3 | tau PFF | 5 | WT | 100 |
| Example 6.4 | tau PFF | 5 | F4 | 100 |

Specifically, each embodiment was prepared by incubating the neuronal cells cultured in Experimental example 6.1 for 48 hours under the conditions in Table 7.

### Experimental Example 6.3 Evaluate In Vitro Efficacy on Primary Hippocampal Neuronal Cells

For each embodiment according to Experimental example 6.2, cell viability was assessed by CCK-8 assay according to Experimental example 1.7.

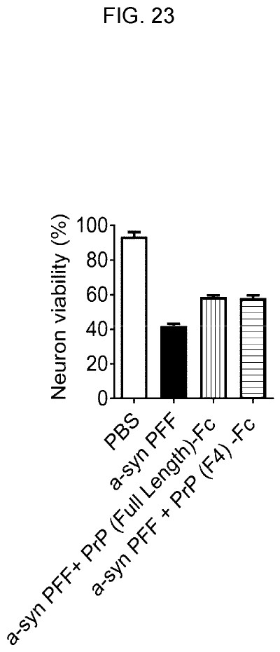

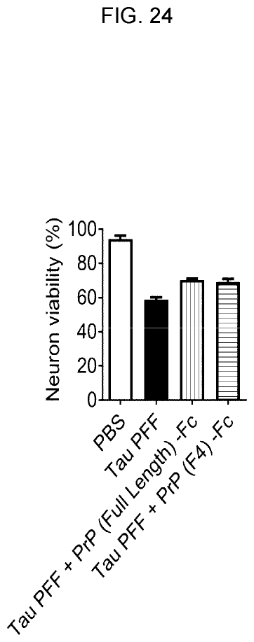

The results of the survivability assessment are shown in FIGS. 23 and 24.

Experiments showed that the above primary hippocampal neuronal cells cultured in media containing the prion-Fc region fusion protein (Example 6.1 through Example 6.4) had higher survival rates than cells cultured in media containing alpha-synuclein PFF, or tau PFF alone (Comparative Example 6.3, and Comparative Example 6.4).

Thus, for primary hippocampal neuronal cells, it can be seen that the prion-Fc region fusion protein of the above embodiment has the effect of eliminating the toxicity of alpha-synuclein PFF and tau protein aggregates, and the prion-Fc region fusion protein comprising a fragment of human prion protein and its variants has the same effect as the prion-Fc region fusion protein comprising wild-type human prion protein.

### Experimental Example 7 Evaluation of In Vitro Efficacy for Human Prion Protein Variants and Fragments 2

### Experimental Example 7.1 Preparation of Embodiments for In Vitro Efficacy Evaluation of Human Prion Protein Variants and Fragments

For in vitro efficacy evaluation of a prion-Fc region fusion protein comprising a fragment of a human prion protein, a variant, or a fragment of a variant, prepare a culture medium comprising a prion-Fc region fusion protein comprising a human prion protein represented by SEQ ID NO. 2 to 15 respectively, referring to Experimental example 1.1, Experimental Example 1.3, and Experimental Example 5.1.

Prepare each embodiment with different culture conditions as described in Experimental example 2.1 by adding alpha-synuclein PFF according to Experimental example 1.4 and culture medium comprising each of the above prion-Fc fusion proteins.

Additionally, a medium to which nothing is added, a medium to which only alpha-synuclein PFF is added, and a medium containing only the culture medium comprising the prion-Fc fusion protein are used as controls.

### Experimental Example 7.2 Viability Assessment for SH-SY5Y Cells (CCK-8 Assay)

For each embodiment according to Experimental example 7.1, evaluate the viability of SH-SY5Y cells according to Experimental example 2.2.

### Experimental Example 7.3 Observe Immunofluorescence Images for SH-SY5Y Cells

For each embodiment according to Experimental example 7.1, acquire and observe immunofluorescence images of SH-SY5Y cells according to Experimental example 2.3.

### Experimental Example 7.4 Western Blot Analysis for Each Embodiment

For each embodiment according to Experimental example 7.1, perform a western blot analysis according to Experimental example 1.9.

### Experimental Example 7.5 Evaluating Microglial Activity

For each embodiment according to Experimental example 7.1, acquire immunofluorescence images of SIM-A9 cells according to Experimental example 3.2, and evaluate the activity of the microglia by observation.

### Experimental Example 8 Evaluation of In Vivo Efficacy for Human Prion Protein Variants and Fragments

### Experimental Example 8.1 Preparation of an Embodiment for In Vivo Efficacy Evaluation of a Prion-Fc Fusion Protein in a Lewy Body Mouse Model

To evaluate the in vivo efficacy of a prion-Fc region fusion protein comprising a fragment, variant, or variant of a human prion protein, an AAV vector capable of expressing a prion-Fc region fusion protein comprising a human prion protein represented by SEQ ID NO. 2 to 15 is prepared respectively, referring to Experimental example 1.1 and Experimental example 5.1.

Referring to Experimental example 4.1, prepare each embodiment by stereotaxically injecting each of the above AAV vectors into a Lewy body model mouse according to Table 4. Here, normal mice and uninjected Lewy body model mice are used as controls.

### Experimental Example 8.2 Pole Test

For each embodiment according to Experimental example 8.1, perform a pole test according to Experimental example 4.2.

### Experimental Example 8.3 Hindlimb Extensor Reflex Test

For each embodiment according to Experimental example 8.1, perform a hind leg extensor reflex test according to Experimental example 4.3.

### Experimental Example 8.4 Observe Immunofluorescence Images of Mouse Brain Tissue

For each embodiment according to Experimental example 8.1, acquire and observe immunofluorescence images of mouse brain tissue according to Experimental example 4.4.

### INDUSTRIAL AVAILABILITY

Disclosed herein are prion-Fc region fusion proteins and vectors capable of expressing them, which can be used for the treatment of diseases associated with neurotoxic protein aggregates.

1. A fusion protein capable of inducing the removal of neurotoxic protein aggregates, represented by [Formula 1]:
hPrP−L1−Fc [Formula 1]
wherein the hPrP is selected from a group of full-length human prion protein, human prion protein fragments, human prion protein variants, and fragment of a human prion protein variants,
wherein the L1 is a linker or absent,
wherein the Fc is the Fc region of human or mouse immunoglobulin G, and
wherein the neurotoxic protein aggregates are one or more selected from a group of alpha-synuclein aggregates, amyloid beta aggregates, tau aggregates, TDP-43 aggregates, and prion aggregates.

2. The fusion protein of claim 1, the hPrP is represented by a sequence selected from the following sequences:
(SEQ ID NO. 1)
MANLGCWMLVLFVATWSDLGLCKKRPKPGGWNTGGSRYPGQGSPGGNRY
PPQGGGGWGQPHGGGWGQPHGGGWGQPHGGGWGQPHGGGWGQGGGTHSQ
WNKPSKPKTNMKHMAGAAAAGAVVGGLGGYMLGSAMSRPIIHFGSDYED
RYYRENMHRYPNQVYYRPMDEYSNQNNFVHDCVNITIKQHTVTTTTKGE
NFTETDVKMMERVVEQMCITQYERESQAYYQRGSSMVLFSSPPVILLIS
FLIFLIVG;
(SEQ ID NO. 30)
PQGGGGWGQPHGGGWGQPHGGGWGQPHGGGWGQPHGGGWGQGGGTHS
QWNKPSKPKTNMKH;
(SEQ ID NO. 31)
PQGGGGWGQPHGGGWGQPHGGGWGQPHGGGWGQPHGGGWGQGGGTHS
QWNKPSKPKTNMKHMAGAAAAGAVVGGLGGYMLGSAM;
(SEQ ID NO. 32)
KKRPKPGGWNTGGSRYPGQGSPGGNRYPPQGGGGWGQPHGGGWGQPHGG
GWGQPHGGGWGQPHGGGWGQGGGTHSQWNKPSKPKTNMKH;
(SEQ ID NO. 33)
KKRPKPGGWNTGGSRYPGQGSPGGNRYPPQGGGGWGQPHGGGWGQPHGG
GWGQPHGGGWGQPHGGGWGQGGGTHSQWNKPSKPKTNMKHMAGAAAAGA
VVGGLGGYMLGSAM;
(SEQ ID NO. 34)
THSQWNKPSKPKTNMKHMAGAAAAGAVVGGLGGYMLGSAM;
(SEQ ID NO. 35)
MANLGCWMLVLFVATWSDLGLCKKRPKPGGWNTGGSRYPGQGSPGGNRY
PPQGGGGWGQPHGGGWGQPHGGGWGQPHGGGWGQPHGGGWGQGGGTHSQ
WNKPSKPKTNMKHMAGAAAAGAVVGGLGVYMLGSAMSRPIIHFGSDYED
RYYRENMHRYPNQVYYRPMDEYSNQNNFVHDCVNITIKQHTVTTTTKGE
NFTETDVKMMERVVEQMCITQYERESQAYYQRGSSMVLFSSPPVILLIS
FLIFLIVG;
(SEQ ID NO. 36)
PQGGGGWGQPHGGGWGQPHGGGWGQPHGGGWGQPHGGGWGQGGGTHS
QWNKPSKPKTNMKHMAGAAAAGAVVGGLGVYMLGSAM;
(SEQ ID NO. 37)
KKRPKPGGWNTGGSRYPGQGSPGGNRYPPQGGGGWGQPHGGGWGQPHGG
GWGQPHGGGWGQPHGGGWGQGGGTHSQWNKPSKPKTNMKHMAGAAAAGA
VVGGLGVYMLGSAM;
(SEQ ID NO. 38)
THSQWNKPSKPKTNMKHMAGAAAAGAVVGGLGVYMLGSAM;
(SEQ ID NO. 39)
MANLGCWMLVLFVATWSDLGLCKKRPKPGGWNTGGSRYPGQGSPGGNRY
PPQGGGGWGQPHGGGWGQPHGGGWGQPHGGGWGQPHGGGWGQGGGTHSQ
WNKPSKPKTNMKHMAGAAAAGAVVGGLGGYVLGSAMSRPIIHFGSDYED
RYYRENMHRYPNQVYYRPMDEYSNQNNFVHDCVNITIKQHTVTTTTKGE
NFTETDVKMMERVVEQMCITQYERESQAYYQRGSSMVLFSSPPVILLIS
FLIFLIVG;
(SEQ ID NO. 40)
KKRPKPGGWNTGGSRYPGQGSPGGNRYPPQGGGGWGQPHGGGWGQPHGG
GWGQPHGGGWGQPHGGGWGQGGGTHSQWNKPSKPKTNMKHMAGAAAAGA
VVGGLGGYVLGSAM;
(SEQ ID NO. 41)
PQGGGGWGQPHGGGWGQPHGGGWGQPHGGGWGQPHGGGWGQGGGTHS
QWNKPSKPKTNMKHMAGAAAAGAVVGGLGGYVLGSAM;
(SEQ ID NO. 42)
THSQWNKPSKPKTNMKHMAGAAAAGAVVGGLGGYVLGSAM;
(SEQ ID NO. 43)
MANLGCWMLVLFVATWSDLGLCKKRPKPGGWNTGGSRYPGQGSPGGNRY
PPQGGGGWGQPHGGGWGQPHGGGWGQPHGGGWGQPHGGGWGQGGGTHSQ
WNKPSKPKTNMKHMAGAAAAGAVVGGLGGYMLGSAMSRPIIHFGSDYED
RYYRENMHRYPNQVYYRPMDEYSNQNNFVHDCVNITIKQHTVTTTTKGE
NFTETDVKMMERVVEQMCITQYKRESQAYYQRGSSMVLFSSPPVILLIS
FLIFLIVG;
and
(SEQ ID NO. 44)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPGK.

3. The fusion protein of claim 1, the L1 is represented by a sequence selected from the following sequences:
ISA; and
(SEQ ID NO. 18)
ISAMVRS.

4. The fusion protein of claim 1, the Fc is the Fc region of human immunoglobulin G.

5. The fusion protein of claim 4, the human immunoglobulin G is selected from a group of IgG1, IgG2, IgG3, and IgG4.

6. The fusion protein of claim 4, the Fc is represented by the sequence:
(SEQ ID NO. 16)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPGK.

7. The fusion protein of claim 1, wherein the fusion protein further comprises a secretion signal peptide, and C-terminus of the secretion signal peptide is linked to N-terminus of the hPrP.

8. The fusion protein of claim 7, wherein the fusion protein further comprises a second linker, and the C-terminus of the secretion signal peptide and the N-terminus of the hPrP are conjugated by the second linker.

9. The fusion protein of claim 7, the second linker is represented by a sequence selected from the following sequences:
ISA; and
(SEQ ID NO. 18)
ISAMVRS.

10. The fusion protein of claim 1, the fusion protein is represented by a sequence selected from following sequences:
(SEQ ID NO. 21)
MANLGCWMLVLFVATWSDLGLCKKRPKPGGWNTGGSRYPGQGSPGGNRY
PPQGGGGWGQPHGGGWGQPHGGGWGQPHGGGWGQPHGGGWGQGGGTHSQ
WNKPSKPKTNMKHMAGAAAAGAVVGGLGGYMLGSAMSRPIIHFGSDYED
RYYRENMHRYPNQVYYRPMDEYSNQNNFVHDCVNITIKQHTVTTTTKGE
NFTETDVKMMERVVEQMCITQYERESQAYYQRGSSMVLFSSPPVILLIS
FLIFLIVGISADKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFF
LYSKLTVDKSRWQQGNVFSCSVMHEALHNHYTQKSLSLSPGK;
(SEQ ID NO. 22)
PQGGGGWGQPHGGGWGQPHGGGWGQPHGGGWGQPHGGGWGQGGGTHS
QWNKPSKPKTNMKHMAGAAAAGAVVGGLGVYMLGSAMISADKTHTCPPC
PAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSC
SVMHEALHNHYTQKSLSLSPGK;
(SEQ ID NO. 23)
PQGGGGWGQPHGGGWGQPHGGGWGQPHGGGWGQPHGGGWGQGGGTHS
QWNKPSKPKTNMKHISADKTHTCPPCPAPELLGGPSVFLFPPKPKDTLM
ISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK;
(SEQ ID NO. 24)
KKRPKPGGWNTGGSRYPGQGSPGGNRYPPQGGGGWGQPHGGGWGQPHGG
GWGQPHGGGWGQPHGGGWGQGGGTHSQWNKPSKPKTNMKHISADKTHTC
PPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNV
FSCSVMHEALHNHYTQKSLSLSPGK;
(SEQ ID NO. 25)
KKRPKPGGWNTGGSRYPGQGSPGGNRYPPQGGGGWGQPHGGGWGQPHGG
GWGQPHGGGWGQPHGGGWGQGGGTHSQWNKPSKPKTNMKHMAGAAAAGA
VVGGLGVYMLGSAMISADKTHTCPPCPAPELLGGPSVFLFPPKPKDTLM
ISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK;
and
(SEQ ID NO. 26)
THSQWNKPSKPKTNMKHMAGAAAAGAVVGGLGVYMLGSAMISADKTHTC
PPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNV
FSCSVMHEALHNHYTQKSLSLSPGK.

11. The fusion protein of claim 7, the fusion protein is represented by a sequence selected from the following sequences:
(SEQ ID NO. 36)
MYRMQLLSCIALSLALVTNSISAMVRSMANLGCWMLVLFVATWSDLGLC
KKRPKPGGWNTGGSRYPGQGSPGGNRYPPQGGGGWGQPHGGGWGQPHGG
GWGQPHGGGWGQPHGGGWGQGGGTHSQWNKPSKPKTNMKHMAGAAAAGA
VVGGLGGYMLGSAMSRPIIHFGSDYEDRYYRENMHRYPNQVYYRPMDEY
SNQNNFVHDCVNITIKQHTVTTTTKGENFTETDVKMMERVVEQMCITQY
ERESQAYYQRGSSMVLFSSPPVILLISFLIFLIVGISADKTHTCPPCPA
PELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSV
MHEALHNHYTQKSLSLSPGK;
(SEQ ID NO. 37)
MYRMQLLSCIALSLALVTNSISAMVRSPQGGGGWGQPHGGGWGQPHGGG
WGQPHGGGWGQPHGGGWGQGGGTHSQWNKPSKPKTNMKHMAGAAAAGAV
VGGLGVYMLGSAMISADKTHTCPPCPAPELLGGPSVFLFPPKPKDTLMI
SRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK;
(SEQ ID NO. 38)
MYRMQLLSCIALSLALVTNSISAMVRSPQGGGGWGQPHGGGWGQPHGGG
WGQPHGGGWGQPHGGGWGQGGGTHSQWNKPSKPKTNMKHISADKTHTCP
PCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVF
SCSVMHEALHNHYTQKSLSLSPGK;
(SEQ ID NO. 39)
MYRMQLLSCIALSLALVTNSISAMVRSKKRPKPGGWNTGGSRYPGQGSP
GGNRYPPQGGGGWGQPHGGGWGQPHGGGWGQPHGGGWGQPHGGGWGQGG
GTHSQWNKPSKPKTNMKHISADKTHTCPPCPAPELLGGPSVFLFPPKPK
DTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLS
PGK;
(SEQ ID NO. 40)
MYRMQLLSCIALSLALVTNSISAMVRSKKRPKPGGWNTGGSRYPGQGSP
GGNRYPPQGGGGWGQPHGGGWGQPHGGGWGQPHGGGWGQPHGGGWGQGG
GTHSQWNKPSKPKTNMKHMAGAAAAGAVVGGLGVYMLGSAMISADKTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGK;
and
(SEQ ID NO. 41)
MYRMQLLSCIALSLALVTNSISAMVRSTHSQWNKPSKPKTNMKHMAGAA
AAGAVVGGLGVYMLGSAMISADKTHTCPPCPAPELLGGPSVFLFPPKPK
DTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLS
PGK.

12. DNA encoding the fusion protein of claim 1.

13. The DNA of claim 12, the fusion protein is represented by a sequence selected from a group of SEQ ID NO: 21 to SEQ ID NO: 26 and SEQ ID NO: 36 to SEQ ID NO: 41.

14. A vector capable of expressing a prion-Fc domain fusion protein comprising:
DNA encoding the fusion protein of claim 1; and a Promoter.

15. The vector of claim 14, wherein the vector is an Adeno-associated virus(AAV) vector.

16.-17. (canceled)

18. A method for treating neurodegenerative diseases, comprising administering to the central nervous system of a subject a composition comprising the fusion protein of claim 1 or the vector of claim 14 and a pharmaceutically acceptable carrier.

19. The method of claim 18, wherein the central nervous system of the subject is a brain tissue of the subject.

20. The method of claim 19, wherein the brain tissue of the subject is selected from a group of Substantia Nigra, Cerebral Ventricle, and Striatum.

21. The method of claim 19, wherein the composition is administered to the subject's central nervous system by a route selected from intracerebral injection and intracerebroventricular injection (ICV).

22.-25. (canceled)


SEQUENCE LISTING (63 sequences)

SEQ ID NO: 1 - protein, 253 aa, homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 2 - protein, 61 aa, Artificial Sequence
  fregment of hPrP
PQGGGGWGQPHGGGWGQPHGGGWGQPHGGGWGQPHGGGWGQGGGTHSQWNKPSKPKTNMKH

SEQ ID NO: 3 - protein, 84 aa, Artificial Sequence
  fregment of hPrP
PQGGGGWGQPHGGGWGQPHGGGWGQPHGGGWGQPHGGGWGQGGGTHSQWNKPSKPKTNMKHMAGAAAAGAVVGGLGGYMLGSAM

SEQ ID NO: 4 - protein, 89 aa, Artificial Sequence
  fregment of hPrP
KKRPKPGGWNTGGSRYPGQGSPGGNRYPPQGGGGWGQPHGGGWGQPHGGGWGQPHGGGWGQPHGGGWGQGGGTHSQWNKPSKPKTNMKH

SEQ ID NO: 5 - protein, 112 aa, Artificial Sequence
  fregment of hPrP
KKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAM

SEQ ID NO: 6 - protein, 40 aa, Artificial Sequence
  fregment of hPrP
THSQWNKPSKPKTNMKHMAGAAAAGAVVGGLGGYMLGSAM

SEQ ID NO: 7 - protein, 253 aa, Artificial Sequence
  variant of hPrP
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGVYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 8 - protein, 84 aa, Artificial Sequence
  fregment of variant of hPrP
PQGGGGWGQPHGGGWGQPHGGGWGQPHGGGWGQPHGGGWGQGGGTHSQWNKPSKPKTNMKHMAGAAAAGAVVGGLGVYMLGSAM

SEQ ID NO: 9 - protein, 112 aa, Artificial Sequence
  fregment of variant of hPrP
KKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGVYMLGSAM

SEQ ID NO: 10 - protein, 40 aa, Artificial Sequence
  fregment of variant of hPrP
THSQWNKPSKPKTNMKHMAGAAAAGAVVGGLGVYMLGSAM

SEQ ID NO: 11 - protein, 253 aa, Artificial Sequence
  variant of hPrP
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYVLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 12 - protein, 112 aa, Artificial Sequence
  fregment of variant of hPrP
KKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYVLGSAM

SEQ ID NO: 13 - protein, 84 aa, Artificial Sequence
  fregment of variant of hPrP
PQGGGGWGQPHGGGWGQPHGGGWGQPHGGGWGQPHGGGWGQGGGTHSQWNKPSKPKTNMKHMAGAAAAGAVVGGLGGYVLGSAM

SEQ ID NO: 14 - protein, 40 aa, Artificial Sequence
  fregment of variant of hPrP
THSQWNKPSKPKTNMKHMAGAAAAGAVVGGLGGYVLGSAM

SEQ ID NO: 15 - protein, 253 aa, Artificial Sequence [sequence omitted]

SEQ ID NO: 16 - protein, 227 aa, Artificial Sequence
  Fc region of hIgG
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 20 aa, Artificial Sequence
  Secretion signal peptide
MYRMQLLSCIALSLALVTNS

SEQ ID NO: 18 - protein, 7 aa, Artificial Sequence
  Linker
ISAMVRS

SEQ ID NO: 19 - protein, 5 aa, Artificial Sequence
  Linker
GGGGS

SEQ ID NO: 20 - protein, 5 aa, Artificial Sequence
  Linker
EAAAK

SEQ ID NO: 21 - protein, 483 aa, Artificial Sequence
  hPrP-Linker-Fc region fusion protein
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVGISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 314 aa, Artificial Sequence
  hPrP-Linker-Fc region fusion protein
PQGGGGWGQPHGGGWGQPHGGGWGQPHGGGWGQPHGGGWGQGGGTHSQWNKPSKPKTNMKHMAGAAAAGAVVGGLGVYMLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 291 aa, Artificial Sequence
  hPrP-Linker-Fc region fusion protein
PQGGGGWGQPHGGGWGQPHGGGWGQPHGGGWGQPHGGGWGQGGGTHSQWNKPSKPKTNMKHISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 319 aa, Artificial Sequence
  hPrP-Linker-Fc region fusion protein
KKRPKPGGWNTGGSRYPGQGSPGGNRYPPQGGGGWGQPHGGGWGQPHGGGWGQPHGGGWGQPHGGGWGQGGGTHSQWNKPSKPKTNMKHISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 342 aa, Artificial Sequence
  hPrP-Linker-Fc region fusion protein
KKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGVYMLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 270 aa, Artificial Sequence
  hPrP-Linker-Fc region fusion protein
THSQWNKPSKPKTNMKHMAGAAAAGAVVGGLGVYMLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 314 aa, Artificial Sequence
  hPrP-Linker-Fc region fusion protein
PQGGGGWGQPHGGGWGQPHGGGWGQPHGGGWGQPHGGGWGQGGGTHSQWNKPSKPKTNMKHMAGAAAAGAVVGGLGGYMLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 342 aa, Artificial Sequence
  hPrP-Linker-Fc region fusion protein
KKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 270 aa, Artificial Sequence
  hPrP-Linker-Fc region fusion protein
THSQWNKPSKPKTNMKHMAGAAAAGAVVGGLGGYMLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 483 aa, Artificial Sequence
  hPrP-Linker-Fc region fusion protein
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGVYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVGISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 483 aa, Artificial Sequence
  hPrP-Linker-Fc region fusion protein
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYVLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVGISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 342 aa, Artificial Sequence
  hPrP-Linker-Fc region fusion protein
KKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYVLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 314 aa, Artificial Sequence
  hPrP-Linker-Fc region fusion protein
PQGGGGWGQPHGGGWGQPHGGGWGQPHGGGWGQPHGGGWGQGGGTHSQWNKPSKPKTNMKHMAGAAAAGAVVGGLGGYVLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 270 aa, Artificial Sequence
  hPrP-Linker-Fc region fusion protein
THSQWNKPSKPKTNMKHMAGAAAAGAVVGGLGGYVLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 483 aa, Artificial Sequence
  hPrP-Linker-Fc region fusion protein
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYKRESQAYYQRGSSMVLFSSPPVILLISFLIFLIVGISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 510 aa, Artificial Sequence
  SS-Linker-hPrP-Linker-Fc region fusion protein
MYRMQLLSCIALSLALVTNSISAMVRSMANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVGISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 341 aa, Artificial Sequence
  SS-Linker-hPrP-Linker-Fc region fusion protein
MYRMQLLSCIALSLALVTNSISAMVRSPQGGGGWGQPHGGGWGQPHGGGWGQPHGGGWGQPHGGGWGQGGGTHSQWNKPSKPKTNMKHMAGAAAAGAVVGGLGVYMLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 318 aa, Artificial Sequence
  SS-Linker-hPrP-Linker-Fc region fusion protein
MYRMQLLSCIALSLALVTNSISAMVRSPQGGGGWGQPHGGGWGQPHGGGWGQPHGGGWGQPHGGGWGQGGGTHSQWNKPSKPKTNMKHISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 346 aa, Artificial Sequence
  SS-Linker-hPrP-Linker-Fc region fusion protein
MYRMQLLSCIALSLALVTNSISAMVRSKKRPKPGGWNTGGSRYPGQGSPGGNRYPPQGGGGWGQPHGGGWGQPHGGGWGQPHGGGWGQPHGGGWGQGGGTHSQWNKPSKPKTNMKHISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 369 aa, Artificial Sequence
  SS-Linker-hPrP-Linker-Fc region fusion protein
MYRMQLLSCIALSLALVTNSISAMVRSKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGVYMLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 297 aa, Artificial Sequence
  SS-Linker-hPrP-Linker-Fc region fusion protein
MYRMQLLSCIALSLALVTNSISAMVRSTHSQWNKPSKPKTNMKHMAGAAAAGAVVGGLGVYMLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 341 aa, Artificial Sequence
  SS-Linker-hPrP-Linker-Fc region fusion protein
MYRMQLLSCIALSLALVTNSISAMVRSPQGGGGWGQPHGGGWGQPHGGGWGQPHGGGWGQPHGGGWGQGGGTHSQWNKPSKPKTNMKHMAGAAAAGAVVGGLGGYMLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 369 aa, Artificial Sequence
  SS-Linker-hPrP-Linker-Fc region fusion protein
MYRMQLLSCIALSLALVTNSISAMVRSKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 297 aa, Artificial Sequence
  SS-Linker-hPrP-Linker-Fc region fusion protein
MYRMQLLSCIALSLALVTNSISAMVRSTHSQWNKPSKPKTNMKHMAGAAAAGAVVGGLGGYMLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 510 aa, Artificial Sequence
  SS-Linker-hPrP-Linker-Fc region fusion protein
MYRMQLLSCIALSLALVTNSISAMVRSMANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGVYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVGISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 510 aa, Artificial Sequence
  SS-Linker-hPrP-Linker-Fc region fusion protein
MYRMQLLSCIALSLALVTNSISAMVRSMANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYVLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVGISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 369 aa, Artificial Sequence
  SS-Linker-hPrP-Linker-Fc region fusion protein
MYRMQLLSCIALSLALVTNSISAMVRSKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYVLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 341 aa, Artificial Sequence
  SS-Linker-hPrP-Linker-Fc region fusion protein
MYRMQLLSCIALSLALVTNSISAMVRSPQGGGGWGQPHGGGWGQPHGGGWGQPHGGGWGQPHGGGWGQGGGTHSQWNKPSKPKTNMKHMAGAAAAGAVVGGLGGYVLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 297 aa, Artificial Sequence
  SS-Linker-hPrP-Linker-Fc region fusion protein
MYRMQLLSCIALSLALVTNSISAMVRSTHSQWNKPSKPKTNMKHMAGAAAAGAVVGGLGGYVLGSAMISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 510 aa, Artificial Sequence
  SS-Linker-hPrP-Linker-Fc region fusion protein
MYRMQLLSCIALSLALVTNSISAMVRSMANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYKRESQAYYQRGSSMVLFSSPPVILLISFLIFLIVGISADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 224 aa, homo sapiens
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 52 - protein, 18 aa, Artificial Sequence
  Secretion signal peptide
MAFLWLLSCWALLGTTFG

SEQ ID NO: 53 - protein, 15 aa, Artificial Sequence
  Secretion signal peptide
MNLLLILTFVAAAVA

SEQ ID NO: 54 - DNA, 759 bp, Artificial Sequence
  hPrP encoding DNA
atggcgaaccttggctgctggatgctggttctctttgtggccacatggagtgacctgggcctctgcaagaagcgcccgaagcctggaggatggaacactgggggcagccgatacccggggcagggcagccctggaggcaaccgctacccacctcagggcggtggtggctgggggcagcctcatggtggtggctgggggcagcctcatggtggtggctgggggcagccccatggtggtggctggggacagcctcatggtggtggctggggtcaaggaggtggcacccacagtcagtggaacaagccgagtaagccaaaaaccaacatgaagcacatggctggtgctgcagcagctggggcagtggtggggggccttggcggctacatgctgggaagtgccatgagcaggcccatcatacatttcggcagtgactatgaggaccgttactatcgtgaaaacatgcaccgttaccccaaccaagtgtactacaggcccatggatgagtacagcaaccagaacaactttgtgcacgactgcgtcaatatcacaatcaagcagcacacggtcaccacaaccaccaagggggagaacttcaccgagaccgacgttaagatgatggagcgcgtggttgagcagatgtgtatcacccagtacgagagggaatctcaggcctattaccagagaggatcgagcatggtcctcttctcctctccacctgtgatcctcctgatctctttcctcatcttcctgatagtggga

SEQ ID NO: 55 - DNA, 252 bp, Artificial Sequence
  hPrP encoding DNA
cctcagggcggtggtggctgggggcagcctcatggtggtggctgggggcagcctcatggtggtggctgggggcagccccatggtggtggctggggacagcctcatggtggtggctggggtcaaggaggtggcacccacagtcagtggaacaagccgagtaagccaaaaaccaacatgaagcacatggctggtgctgcagcagctggggcagtggtggggggccttggcgtttacatgctgggaagtgccatg

SEQ ID NO: 56 - DNA, 183 bp, Artificial Sequence
  hPrP encoding DNA
cctcagggcggtggtggctgggggcagcctcatggtggtggctgggggcagcctcatggtggtggctgggggcagccccatggtggtggctggggacagcctcatggtggtggctggggtcaaggaggtggcacccacagtcagtggaacaagccgagtaagccaaaaaccaacatgaagcac

SEQ ID NO: 57 - DNA, 267 bp, Artificial Sequence
  hPrP encoding DNA
aagaagcgcccgaagcctggaggatggaacactgggggcagccgatacccggggcagggcagccctggaggcaaccgctacccacctcagggcggtggtggctgggggcagcctcatggtggtggctgggggcagcctcatggtggtggctgggggcagccccatggtggtggctggggacagcctcatggtggtggctggggtcaaggaggtggcacccacagtcagtggaacaagccgagtaagccaaaaaccaacatgaagcac

SEQ ID NO: 58 - DNA, 336 bp, Artificial Sequence
  hPrP encoding DNA
aagaagcgcccgaagcctggaggatggaacactgggggcagccgatacccggggcagggcagccctggaggcaaccgctacccacctcagggcggtggtggctgggggcagcctcatggtggtggctgggggcagcctcatggtggtggctgggggcagccccatggtggtggctggggacagcctcatggtggtggctggggtcaaggaggtggcacccacagtcagtggaacaagccgagtaagccaaaaaccaacatgaagcacatggctggtgctgcagcagctggggcagtggtggggggccttggcgtttacatgctgggaagtgccatg

SEQ ID NO: 59 - DNA, 120 bp, Artificial Sequence
  hPrP encoding DNA
acccacagtcagtggaacaagccgagtaagccaaaaaccaacatgaagcacatggctggtgctgcagcagctggggcagtggtggggggccttggcgtttacatgctgggaagtgccatg

SEQ ID NO: 60 - DNA, 469 bp, homo sapiens
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 61 - DNA, 60 bp, homo sapiens
atgtacaggatgcaactcctgtcttgcattgcactaagtcttgcacttgtcacgaattcg

SEQ ID NO: 62 - DNA, 681 bp, Artificial Sequence
  IgG-Fc encoding DNA
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 63 - DNA, 726 bp, Artificial Sequence
  eGFP encoding DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggataa